(19) United States
(12) Patent Application Publication
(10) Pub. No.: US 2026/0137767 A1
(43) Pub. Date: May 21, 2026

(54) LIPOPEPTIDE BUILDING BLOCKS AND SYNTHETIC VIRUS-LIKE PARTICLES

(71) Applicants: VIROMETIX AG, Schlieren (CH); UNIVERSITÄT ZÜRICH, Zürich (CH)

(72) Inventors: Arin GHASPARIAN, Zürich (CH); John A. ROBINSON, Kemptthal (CH)

(21) Appl. No.: 19/364,474

(22) Filed: Oct. 21, 2025

Related U.S. Application Data
(63) Continuation of application No. 17/415,305, now Pat. No. 12,465,633.

(30) Foreign Application Priority Data
Dec. 20, 2018 (EP) 18214485.7

(51) Int. Cl. A61K 39/12; A61K 39/00; A61P 31/14; C07K 14/00; C12N 7/00

(52) U.S. Cl. CPC A61K 39/12; A61P 31/14; C07K 14/001; C12N 7/00; A61K 2039/5258; A61K 2039/6018; C12N 2760/18523; C12N 2760/18534

(57) ABSTRACT

The present invention relates to a lipopeptide building block consisting of
(i) a peptide moiety comprising a coiled coil peptide chain segment, wherein said coiled coil peptide chain segment comprises 3 to 8 repeat units, and wherein said repeat unit consists of the sequence IEKKIE-X0 (SEQ ID NO:58), wherein X0 represents an amino acid, and wherein preferably said repeat unit consists of the sequence selected from IEKKIEG (SEQ ID NO:59), IEKKIEA (SEQ ID NO:12) or IEKKIES (SEQ ID NO:13), and wherein further preferably said repeat unit consists of the sequence IEKKIES (SEQ ID NO:13);
(ii) a lipid moiety comprising, preferably consisting of, the formula LM-I

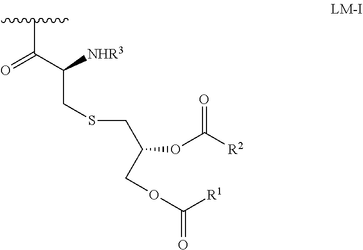

wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁;
and wherein said lipid moiety is linked to said peptide moiety, wherein the wavy line in formula LM-I indicates the linkage site to said peptide moiety, and wherein preferably said lipid moiety is linked to the N-terminus of said peptide moiety, as well as conjugates comprising said lipopeptide building blocks to which antigens are coupled, bundles of such conjugates, synthetic virus-like particles (SVLPs) comprising at least one bundle of conjugates and pharmaceutical compositions comprising the same. The present invention further relates to said conjugates, bundles of conjugates, said SVLPs and said pharmaceutical compositions for use as a medicament, as vaccines and for use in methods of preventing or treating a disease, preferably selected from infectious diseases, allergies and cancer, and generally to efficiently induce antigen specific immune responses.

### REFERENCE TO ELECTRONIC SEQUENCE LISTING

The application contains a Sequence Listing which has been submitted electronically in XML format and is hereby incorporated by reference in its entirety. Said XML copy, created on Jan. 29, 2026, is named 0192-0136US2.XML and is 182,343 bytes in size.

The present invention relates to lipopeptide building blocks, conjugates comprising said lipopeptide building blocks to which antigens are coupled, bundles of such conjugates, synthetic virus-like particles (SVLPs) comprising at least one bundle of conjugates and pharmaceutical compositions comprising the same. The present invention further relates to said conjugates, bundles of conjugates, said SVLPs and said pharmaceutical compositions for use as a medicament, as vaccines and for use in methods of preventing or treating a disease, preferably selected from infectious diseases, allergies and cancer, and generally to efficiently induce antigen specific immune responses.

### RELATED ART

Synthetic virus-like particle (SVLP) have recently attracted a great deal of interest, in particular, SVLP-based vaccine candidates using synthetic lipopeptide building blocks to which antigens are conjugated and which spontaneously self-assemble into highly immunogenic nanoparticles of 20-30 nm and comprising around 60-80 copies of said lipopeptide building blocks. Since said antigens are displayed across the outer surface of the nanoparticle and SVLP, respectively, strong immune responses against the antigens, in particular, B cell responses are induced. Importantly, SVLPs contain no genetic information and cannot replicate in cells. Typically, said lipopeptide building blocks comprise coiled-coil peptide sequences as well as T-helper cell epitopes (A, Ghasparian and John A. Robinson in . . . Eds. . . . ; A. Ghasparian, et al., ChemBioChem 2011, 12, 100-109; T. Riedel, et al., ChemBioChem 2011, 12, 2829; R. Sharma, et al., PLoS One 2012, 7, e43248; WO2008/068017; WO2015/082501). Coiled-coil designs are known by the skilled person in the art and have been reviewed (Woolfson, D. N., Adv. Prot. Chem., 2005, 70, 79-112; Parry, D. A. D., et al., Advancs in Protein Chemistry, 2005, 70).

Specifically engineered SVLPs and their use as vaccines have recently been reported and described a lipopeptide building block containing the coiled coil (IEKKIEA)₄ known to form trimeric parallel helical bundles fused to the promiscuous CD4+ T-helper epitope (IEKKIAKMEKASSVFNVVNS) identified in the circumsporozoite (CS) protein of the malaria parasite Plasmodium falciparum has been described (A. Ghasparian, et al., ChemBioChem 2011, 12, 100-109). The T-helper epitope corresponds (with two Cys-to-Ala substitutions) to residues 379-398 of the CS protein, and is recognized by mouse and human T cells in association with a wide variety of different MHC class II molecules (J. Kilgus, et al., J. Immunol. 1991, 146, 307; F. Sinigaglia, et al., Nature 1988, 336, 778). Said described lipopeptide building block is completed by addition of KKKC at the C-terminus, to allow conjugation of a B-cell epitope through the cysteine thiol and by adding a lipid moiety at the N-terminus, comprising either a phospholipid related to phosphatidylethanolamine, or Pam₂Cys or Pam₃Cys moieties (A. Ghasparian, et al., ChemBioChem 2011, 12, 100-109). The Pam₃Cys moiety, i.e. tripalmitoyl-S-glyceryl cysteine (N-palmitoyl-S-(2,3-bis-(O-palmitoyloxy)-propyl)-cysteinyl-) and the Pam₂Cys moiety, i.e. dipalmitoyl-S-glyceryl cysteine (2,3-bis-(O-palmitoyloxy)-propyl)-cysteinyl-), are known to skilled person in the art and have been well described (Ghielmetti, M., et al., Immunobiology, 2005, 210, 211-215; Reutter, F., et al., J. Pept. Res., 2005, 65, 375-383; Buwitt-Beckmann, U., et al., Eur. J. Immunol., 2005, 35, 1-8).

Despite the recent advances in the development of lipopeptide building blocks and SVLPs, in particular, SVLP-based vaccine candidates, respectively, there is still a need for further and even more effective lipopeptide building blocks and SVLPs, respectively, specifically in order to further enhance the quality of the induced immune responses.

### SUMMARY OF THE INVENTION

We have now surprisingly found that lipopeptide building blocks comprising Pam₂Cys or Pam₃Cys moieties with the (R)-configuration at the 2-propyl carbon atom and further comprising as coiled coil peptide chain segment several units of the sequence IEKKIE-X0 with preferably X0 being Gly, Ala or Ser, most preferably Ser, showed increased avidity of the antibodies generated to antigens linked to the inventive lipopeptide building blocks and comprised by the inventive conjugates or SVLPs, respectively.

Thus, in a first aspect, the present invention provides a lipopeptide building block consisting of
(i) a peptide moiety comprising a coiled coil peptide chain segment, wherein said coiled coil peptide chain segment comprises 3 to 8 repeat units, and wherein said repeat unit consists of the sequence IEKKIE-X0 (SEQ ID NO:58), wherein X0 represents an amino acid, and wherein preferably said repeat unit consists of the sequence selected from IEKKIEG (SEQ ID NO:59), IEKKIEA (SEQ ID NO:12) or IEKKIES (SEQ ID NO:13), and wherein further preferably said repeat unit consists of the sequence IEKKIES (SEQ ID NO:13);
(ii) a lipid moiety comprising, preferably consisting of, the formula LM-I

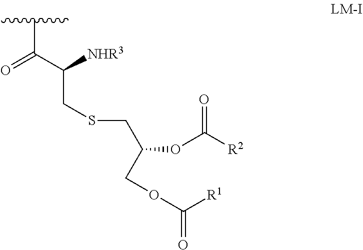

wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁;
and wherein said lipid moiety is linked to said peptide moiety, wherein the wavy line in formula LM-I indicates the linkage site to said peptide moiety, and wherein preferably said lipid moiety is linked to the N-terminus of said peptide moiety.

In a further aspect, the present invention provides a conjugate comprising (a) a lipopeptide building block of the present invention and (b) an antigen, wherein said antigen is connected, directly or via a linker, to said lipopeptide building block.

In a further aspect, the present invention provides a conjugate comprising (a) a lipopeptide building block of the present invention and (b) an antigen, wherein said antigen is connected, directly or via a linker, to said lipopeptide building block.

In another aspect, the present invention provides for a bundle of conjugates comprising 2, 3, 4, 5, 6 or 7, preferably 2, 3, 4 or 5, more preferably 3, of the inventive conjugate.

In another aspect, the present invention provides for a synthetic virus-like particle comprising at least one bundle of conjugates of the present invention.

In another aspect, the present invention provides for a pharmaceutical composition comprising an immunologically effective amount of the conjugate of the present invention or the synthetic virus like particle of the present invention, together with a pharmaceutically acceptable diluent, carrier or excipient, wherein preferably said pharmaceutical composition is a vaccine.

In again another aspect, the present invention provides for the conjugate of the present invention or the synthetic virus like particle of the present invention for use as a medicament.

In again another aspect, the present invention provides for the conjugate of the present invention or the synthetic virus like particle of the present invention for use in a method for preventing a disease or for reducing the risk of a disease, wherein further preferably said disease is an infectious disease, a cancer or an allergy, and again more preferably wherein said disease is an respiratory syncytial virus (RSV) infection.

In again another aspect, the present invention provides for the conjugate of the present invention or the synthetic virus like particle of the present invention for use in a method for preventing a disease or for reducing the risk of a disease or for treating a disease, wherein further preferably said disease is an infectious disease, a cancer or an allergy, and again more preferably wherein said disease is an respiratory syncytial virus (RSV) infection. In again another aspect, the present invention provides for the conjugate of the present invention or the synthetic virus like particle of the present invention for use in a method for preventing of an infectious disease or reducing the risk of an infectious disease, preferably for use in a method for preventing or reducing the risk of an infectious disease associated with or caused by a respiratory syncytial virus.

Further aspects and embodiments of the present invention will become apparent as this description continues.

### BRIEF DESCRIPTION OF FIGURES

FIG. 1 shows mean avidity indexes of conjugates 44, 45 and 46 as determined in immunogenicity studies as described in Example 4.

### DETAILED DESCRIPTION OF THE INVENTION

Unless defined otherwise, all technical and scientific terms used herein have the same meaning as commonly understood by one of ordinary skill in the art to which this invention belongs.

Throughout this specification and the claims, which follow, unless the context requires otherwise, the term “comprise” and its variations such as “comprises” and “comprising” etc., are to be understood as a non-exhaustive wording and imply the inclusion of a stated feature or element but not the exclusion of any other feature or element. The term “comprise” and its variations cover the term “consisting of”. As used in this specification and the appended claims, the singular forms “a”, “an”, and “the” include plural referents, unless the content clearly dictates otherwise. The terms “reduce”, “inhibit” or “decrease”, as used herein, include a just detectable reduction but also a reduction down to zero (reduction by 100%).

A peptide or peptide moiety, as defined herein, is any peptide-bond-linked chain of amino acids, regardless of length, secondary and tertiary structure, number of subunits or post-translational modification. Thus, the term “peptide” of “peptide moiety” is to be understood as covering the terms “polypeptide”, “protein”, “amino acid chain” and “polypeptide chain”. Amino acids included in the peptide of the invention are proteinogenic, non-proteinogenic and synthetic amino acids. Peptides can be an open linear peptide chain or cyclic peptides; and may include at least one chemical modification, such as lipidation, glycosylation and phosphorylation. Peptides can be produced by chemical synthesis, RNA translation and/or recombinant processes.

The term “cyclic peptide”, as used herein, refers to a peptide in which the amino acid chain forms at least one ring structure by a covalent bond. The cyclic peptide of the invention comprises two ring structures each formed by a disulfide bond: Side chains of cysteines C₄ and C₂₅ are linked forming a first disulfide bond, and side chains of cysteines C₈ and C₂₁ are linked forming a second disulfide bond.

The term “amino acid”, as used herein, refers to organic compounds containing the functional groups amine (—NH2) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids.

The term “amino acid”, as used herein, also includes unnatural amino acids that are chemically synthesized; alpha- (α-), beta- (β-), gamma- (γ-) and delta- (δ-) etc. amino acids as well as mixtures thereof in any ratio; and, if applicable, any isomeric form of an amino acid, i.e. its D-stereoisomers (labelled with a lower-case initial letter) and L-stereoisomers (labelled with a capital initial letter) (alternatively addressed by the (R) and (S) nomenclature) as well as mixtures thereof in any ratio, preferably in a racemic ratio of 1:1. Amino acids in this invention are preferably in L-configuration, unless mentioned specifically as D-configuration. The term “D-stereoisomer”, “L-stereoisomer”, “D-amino acid” or “L-amino acid” refers to the chiral alpha carbon of the amino acids. Amino acid can include one or more modifications and/or attached groups, for example protecting groups used for peptide synthesis, such as Boc, Fmoc or both. The term “deletion” refers herein to a position in an amino acid sequence that is not occupied by an amino acid. In a preferred embodiment of the present invention, an amino acid is a naturally occurring amino acid, wherein said naturally occurring amino acid is in its L-configuration, in its D-configuration, or in a mixture of any ratio of said L-configuration and D-configuration. In a further preferred embodiment of the present invention, an amino acid is a naturally occurring amino acid, wherein said naturally occurring amino acid is in its L-configuration.

The term “antigen” as used herein, should refer to molecules capable of being bound by an antibody. The antigen may comprise a peptide, a protein or an epitope mimetic having one or more B-cell epitopes that are to be used to elicit an antigen-specific humoral immune response in an animal. Alternatively, the antigen may comprise a hapten or a carbohydrate. Suitable peptide and protein antigens comprise up to 150 amino acids and include glycopeptides and glycoproteins. Peptide and protein sequences may be so chosen as to elicit an immune response, for example against one or more varieties of infectious agents. Such antigens are well known in the art. An epitope mimetic is a molecule mimicking a natural peptidic or carbohydrate epitope, including peptidic compounds containing one or more non-natural amino acids, e.g. D-amino acids, β-amino acids, γ-amino acids, α-amino acids, or ε-amino acids, and other replacements known in the art of epitope mimics. Preferred are conformationally constrained peptidomimetics, which are fixed in a protein-like conformation. Haptens refer to organic compounds with a molecular weight of less than 3′000, which do not elicit humoral immune responses by themselves, but will elicit an immune response once attached to a carrier. Exemplary haptens include drugs, hormones, toxins and carbohydrates.

The term “N-terminus”, as used herein, refers to an end of a peptide having a free (—NH₂) or modified amino or amine group. Preferred N-terminal modifications are those that protect the N-terminus from proteolytic degradation. N-terminal modifications in accordance with the invention include but are not limited to acetylation, attachment of at least one polymer, preferably polyethylene glycol (PEGylation) or poly(lactic acid), or attachment of at least one amino acid, preferably of at least one D-amino acid, or attachment of at least one compound, such as a cell penetrating peptide, nucleic acid, a carbamate, such as fluorenylmethoxycarbamate or benzyloxy carbamate, aldehyde, hydrazinonicotinic acid, 4-formyl benzamide, methyl, myristoyl, prenyl group, palmitoyl, ubiquitin, 7-methoxycoumarin acetic acid (Mca), dansyl, formyl, 4-diniphenyl, pyroglutamyl, urea, carbamate, sulphonamide, alkylamine, fatty acids, such as palmitic acids, radioligand, quencher, fluorescein or another dye or label such as biotin.

The term “C-terminus”, as used herein, refers to an end of a peptide having a free (—COOH) or modified carboxyl group. Preferred C-terminal modifications are those that protect the C-terminus from proteolytic degradation. C-terminal modifications in accordance with the invention include but are not limited to amidation or attachment of at least one amino acid, preferably of at least one D-amino acid, or attachment of at least one compound, such as a cell penetrating peptide, nucleic acid, polyethylene glycol (PEGylation), thiol, ester, aldehyde, sulphonamide, pNA (para-nitroanilide), Amc (7-amino-4-methylcoumarinyl), hydrazide, hydroxamic acid, chloromethyl ketone, biotin, radioligand, quencher, Abz or other dyes and labels. Herein and by general convention, peptide sequences are written from N-terminal on the left to C-terminal on the right (according to the direction of translation).

As used herein, the term “coiled coil peptide chain segment” is a sequence of a peptide chain capable of forming a coiled coil (super coil) with at least one other coiled coil peptide chain segment. A coiled coil is a peptide structure in which at least two coiled coil peptide chain segments, each having preferably an alpha helical secondary structure, are associated into a bundle. Coiled coil peptide chain segments of the invention contain multiple repeat units, typically and preferably consecutively linked to each other. The repeat units of the coiled coil peptide chain segment may be identical or may be different, e.g. may contain at least one discontinuity, such as an insertion, deletion or exchange of at least one, preferably exactly 1, 2, 3 or 4 amino acids within the repeat unit.

In a first aspect, the invention provides a lipopeptide building block consisting of
(i) a peptide moiety comprising a coiled coil peptide chain segment, wherein said coiled coil peptide chain segment comprises 3 to 8 repeat units, and wherein said repeat unit consists of the sequence IEKKIE-X0 (SEQ ID NO:58), wherein X0 represents an amino acid, and wherein preferably said repeat unit consists of the sequence selected from IEKKIEG (SEQ ID NO:59), IEKKIEA (SEQ ID NO:12) or IEKKIES (SEQ ID NO:13), and wherein further preferably said repeat unit consists of the sequence IEKKIES (SEQ ID NO:13);
(ii) a lipid moiety comprising, preferably consisting of, the formula LM-I

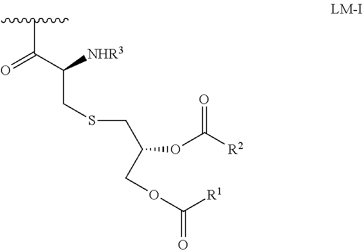

wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁;
and wherein said lipid moiety is linked to said peptide moiety, wherein the wavy line in formula LM-I indicates the linkage site to said peptide moiety, and wherein preferably said lipid moiety is linked to the N-terminus of said peptide moiety.
   In a preferred embodiment, said coiled coil peptide chain segment of said peptide moiety consists of 3 to 8 repeat units including 3, 4, 5, 6, 7, 8 repeat units, more preferably four repeat units. The upper number of repeat units in the peptide moiety influences the stability of the coiled coil. In a preferred embodiment, said coiled coil peptide chain segment of said peptide moiety comprises or preferably consists of 4 repeat units. In a preferred embodiment, said coiled coil peptide chain segment of said peptide moiety consists of 4 repeat units. In a preferred embodiment, said 4 repeat units are consecutively linked to each other.

Coiled coil peptide chain segments of the invention are based on canonical repeat units, typically and preferably canonical tandem heptad repeats that form right-handed amphipathic alpha-helices, which then assemble to form helical bundles with left-handed coiled coils.

Repeat units of coiled coil peptide chain segments have a sequence with a certain number of amino acids, wherein the positions of the amino acids are traditionally labelled as lowercase letters. Design rules are discussed in more detail, for example, in Woolfson, D. N., Adv. Prot. Chem. 2005, 70, 79-112.

In the invention, said repeat unit of the coiled coil peptide chain segments consists of seven amino acids, wherein the seven amino acid positions are designated with letters a, b, c, d, e, f and g. In a preferred embodiment, said heptad motif consist of the sequence IEKKIE-X0 (SEQ ID NO:58), wherein X0 represents an amino acid. In a preferred embodiment, said repeat unit consists of the sequence IEKKIE-X0 (SEQ ID NO:58), wherein X0 represents an amino acid provided that said X0 is not proline. In another preferred embodiment, said repeat unit consists of the sequence IEKKIE-X0 (SEQ ID NO:58), wherein X0 represents an amino acid, wherein said amino acid is a naturally occurring amino acid, wherein said naturally occurring amino acid is in its L-configuration, in its D-configuration, or in a mixture of any ratio thereof, provided that said amino acid is not proline. In another preferred embodiment, said repeat unit consists of the sequence IEKKIE-X0 (SEQ ID NO:58), wherein X0 represents an amino acid, wherein said amino acid is a naturally occurring amino acid in its L-configuration.

In a preferred embodiment, said repeat unit consists of the sequence selected from IEKKIEG (SEQ ID NO:59), IEKKIEA (SEQ ID NO:12) or IEKKIES (SEQ ID NO:13). In a preferred embodiment, said repeat unit consists of the sequence selected from IEKKIEA (SEQ ID NO: 12) or IEKKIES (SEQ ID NO: 13). In a preferred embodiment, said repeat unit consists of the sequence IEKKIEG (SEQ ID NO:59). In a preferred embodiment, said repeat unit consists of the sequence IEKKIEA (SEQ ID NO:12). In a very preferred embodiment, said repeat unit consists of the sequence IEKKIES (SEQ ID NO:13).

In a preferred embodiment, said coiled coil peptide chain segment comprises, or preferably consists of, the sequence selected from (IEKKIEG)₄ (SEQ ID NO:60), (IEKKIEA)₄ (SEQ ID NO:61) or (IEKKIES)₄ (SEQ ID NO:62). In a preferred embodiment, said coiled coil peptide chain segment consists of the sequence selected from (IEKKIEG)₄ (SEQ ID NO:60), (IEKKIEA)₄ (SEQ ID NO:61) or (IEKKIES)₄ (SEQ ID NO:62). In a preferred embodiment, said coiled coil peptide chain segment comprises, or preferably consists of, the sequence (IEKKIEG)₄ (SEQ ID NO:60). In a preferred embodiment, said coiled coil peptide chain segment consists of, the sequence (IEKKIEG)₄ (SEQ ID NO:60).

In a preferred embodiment, said coiled coil peptide chain segment comprises, or preferably consists of, the sequence (IEKKIEA)₄ (SEQ ID NO:61). In a preferred embodiment, said coiled coil peptide chain segment consists of, the sequence (IEKKIEA)₄ (SEQ ID NO:61).

In a very preferred embodiment, said coiled coil peptide chain segment comprises, or preferably consists of, the sequence (IEKKIES)₄ (SEQ ID NO:62). In a very preferred embodiment, said coiled coil peptide chain segment consists of the sequence (IEKKIES)₄ (SEQ ID NO:62).

In a preferred embodiment, said R¹ and R² are independently —C₁₁H₂₃, —C₁₃H₂₇ or —C₁₅H₃₁. In a very preferred embodiment, said R¹ and R² are —C₁₅H₃₁. In a preferred embodiment, said R³ is H or —C(O)C₁₅H₃₁.

In a preferred embodiment, said R¹ and R² are independently —C₁₁H₂₃, —C₁₃H₂₇ or —C₁₅H₃₁, and R³ is hydrogen or —C(O)C₁₁₋₁₅alkyl. In a very preferred embodiment, said R¹ and R² are —C₁₅H₃₁, and R³ is hydrogen or —C(O)C₁₁₋₁₅alkyl.

In a preferred embodiment, said R¹ and R² are independently —C₁₁H₂₃, —C₁₃H₂₇ or —C₁₅H₃₁, and R³ is H or —C(O)C₁₅H₃₁. In a very preferred embodiment, said R¹ and R² are —C₁₅H₃₁, and R³ is H or —C(O)C₁₅H₃₁.

In a preferred embodiment, said lipid moiety is linked to the N-terminus of said peptide moiety. This conveniently allows that said linking can be performed on-resin after assembly of the peptide chain of said peptide moiety by solid phase peptide synthesis. Linking of said lipid moiety to the C-terminus of said peptide moiety is also encompassed within the present invention and is possible using linkage chemistry known by the skilled person in the art.

In a preferred embodiment, said peptide moiety has a length of 12 to 200 amino acids, more preferably of 21 to 120 amino acids, again more preferably of 21 to 80 amino acids, again more preferably of 21 to 70 amino acids again more preferably of 21 to 60 amino acids again more preferably of 21 to 50 amino acids, again more preferably said peptide moiety has a length of 28 to 48 amino acids. Preferred peptide moieties are non-human sequences to avoid the risk of autoimmune disorders when applied in the vaccination of humans.

A preferred lipid moiety is di-palmitoyl-S-glycerylcysteinyl (Pam₂Cys) or tripalmitoyl-S-glyceryl cysteine (Pam₃Cys), both with the R-configuration at the chiral 2-propyl carbon atom and the R-configuration of the chiral carbon of the cysteinyl moiety.

In a preferred embodiment, the lipid moiety is linked to the peptide moiety, either directly or via a coupling moiety. Preferably, the lipid moiety is linked to the peptide moiety at or near one terminus, i.e. the N-terminus or the C-terminus, preferably the N-terminus. In a preferred embodiment, the lipid moiety is linked to the first, second, third, fourth or fifth amino acid of the peptide moiety, calculated from the N-terminus or C-terminus of the peptide moiety. The lipid moiety may be linked, directly or through a coupling moiety, to the backbone or to the side chain of one of the amino acids of the peptide moiety, preferably said amino acid is near to the terminus, more preferably it is the first, second, third, fourth or fifth amino acid of the peptide moiety.

The lipid moiety is attached to the peptide moiety directly, or via a coupling moiety. If the peptide moiety and the lipid moiety are directly linked, this is preferably accomplished through an amide bond between a lipid moiety carbonyl function and an amino function, e.g. the N-terminal amino function, of the peptide moiety. It will be apparent to the skilled person in the art that a large variety of suitable coupling moieties and coupling strategies exist, which include but are not limited to linkers based on dicarboxylic acid derivatives, linkers containing one or multiple ethylene glycol units, amino acid residues (including alpha-, beta-, gamma-, omega-amino acids), or sugar (carbohydrate) units, or containing heterocyclic rings.

In a preferred embodiment, said lipid moiety comprises, preferably consists of, the formula LM-I*

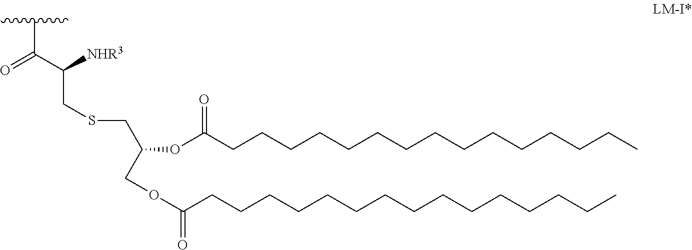

wherein R³ is hydrogen or —C(O)C₁₁₋₁₅alkyl, preferably H or —C(O)C₁₅H₃₁; wherein preferably said lipid moiety is linked to the N-terminus of said peptide moiety.

In a preferred embodiment, said lipid moiety consists of the formula LM-I*, wherein R³ is hydrogen or —C(O)C₁₁₋₁₅alkyl. In a preferred embodiment, said lipid moiety comprises, preferably consists of, the formula LM-I*, wherein R³ is H or —C(O)C₁₅H₃₁. In a preferred embodiment, said lipid moiety comprises, preferably consists of, the formula LM-I*, wherein R³ is H or —C(O)C₁₅H₃₁ and wherein said lipid moiety is linked to the N-terminus of said peptide moiety. In a preferred embodiment, said lipid moiety consists of, the formula LM-I*

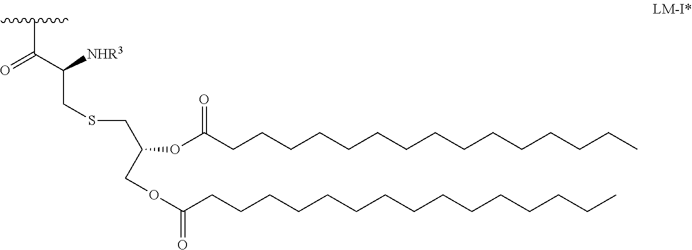

wherein R³ is H or —C(O)C₁₅H₃₁ and wherein preferably said lipid moiety is linked to the N-terminus of said peptide moiety.

In a very preferred embodiment, said lipid moiety comprises, preferably consists of, the formula LM-I*1 or LM-I*2. In a very preferred embodiment, said lipid moiety consists of the formula LM-I*1 or LM-I*2.

In a very preferred embodiment, said lipid moiety consists of the formula LM-I*1.

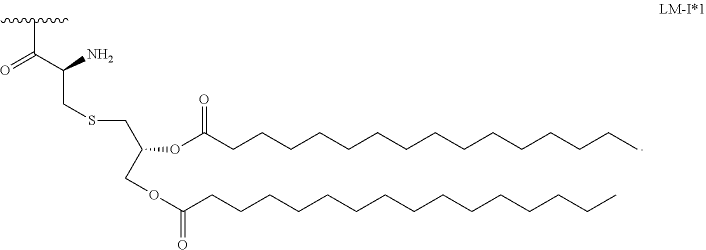 [SMILES: *C(=O)[C@@H](N)CSC[C@@H](COC(=O)CCCCCCCCCCCCCCC)OC(=O)CCCCCCCCCCCCCCC]

In a very preferred embodiment, said lipid moiety consists of the formula LM-I*2.

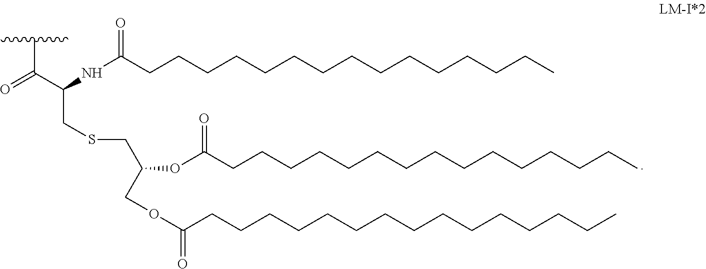 [SMILES: *C(=O)[C@H](CSC[C@@H](COC(=O)CCCCCCCCCCCCCCC)OC(=O)CCCCCCCCCCCCCCC)NC(=O)CCCCCCCCCCCCCCC]

Very preferred lipid moieties of the present invention are, thus, (R,R)-Pam₃Cys LM-I*2, i.e. tripalmitoyl-S-glyceryl cysteine (N-palmitoyl-S-(2,3-bis-(O-palmitoyloxy)-(2R)-propyl)-(R)-cysteinyl-) and (R,R)-Pam₂Cys LM-I*1, i.e. dipalmitoyl-S-glyceryl cysteine (2,3-bis-(O-palmitoyloxy)-(2R)-propyl)-(R)-cysteinyl-).

Thus, in a further very preferred embodiment, said lipid moiety is N-α-Palmitoyl-S-[2,3-bis(palmitoyloxy)-(2R)-propyl]-(R)-cysteine or S-[2,3-bis(palmitoyloxy)-(2R)-propyl]-(R)-cysteine, thus LM-I*1.

In a further preferred embodiment, said peptide moiety comprises (i) an N-terminal amino acid sequence, wherein said N-terminal amino acid sequence comprises or preferably consists of fibroblast-stimulating lipopeptide FSL-1 (S-(2,3-bispalmitoyloxypropyl)- or PAM2-Cys-Gly-Asp-Pro-Lys-His-Pro-Lys-Ser-Phe; SEQ ID NO:7), FSL-2 (S-(2,3-bispalmitoyloxypropyl)- or PAM2-Cys-Gly-Asp-Pro-Lys-His-Pro-Lys-Ser-Arg; SEQ ID NO:8), FSL-3 (S-(2,3-bisstearyloxypropyl)-Cys-Gly-Asp-Pro-Lys-His-Pro-Lys-Ser-Phe; SEQ ID NO:9), Mycoplasma fermentans-derived peptide MALP-2 (S-(2,3-bispalmitoyloxypropyl)- or PAM2-Cys-Gly-Asn-Asn-Asp-Glu-Ser-Asn-Ile-Ser-Phe-Lys-Glu-Lys; SEQ ID NO:10), or GG; and/or GX where X is Asx or Ser and/or (ii) a C-terminal amino acid sequence, wherein said C-terminal amino acid sequence comprises or preferably consists of a sequence recognized by an enzyme as cleavage site; wherein preferably said C-terminal amino acid sequence comprises or preferably consists of sequence KKiKCa (SEQ ID NO: 11) or wherein preferably said C-terminal amino acid sequence is an amino acid sequence of consecutive 5 amino acids.

In a preferred embodiment, said lipid moiety and said peptide moiety are directly linked. In a preferred embodiment, said lipid moiety and said peptide moiety are linked via a coupling moiety. In a preferred embodiment, said lipid moiety and said peptide moiety are linked via a coupling moiety, wherein said coupling moiety is an amino acid linker consisting of 2-15 amino acids. Examples hereto include the amino acid linker sequences comprised by FSL-1, FSL-2, FSL-3, PAM2 or MALP-2 moieties. In a preferred embodiment, said lipid moiety and said peptide moiety are linked via a coupling moiety, wherein said coupling moiety is an amino acid linker consisting of 2-10 amino acids. In a preferred embodiment, said lipid moiety and said peptide moiety are linked via a coupling moiety, wherein said coupling moiety is an amino acid linker consisting of 2-5 amino acids. In a preferred embodiment, said lipid moiety and said peptide moiety are linked via a coupling moiety, wherein said coupling moiety is an amino acid linker, wherein said amino acid linker is a Gly-Gly moiety. In a preferred embodiment, said lipid moiety and said peptide moiety are linked via a coupling moiety, wherein said coupling moiety is a Gly-Gly moiety.

In a preferred embodiment, said direct linking of said lipid moiety and said peptide moiety is by way of an amide bond between a carbonyl function of said lipid moiety and an amino function of said peptide moiety.

In a preferred embodiment, said linking of said lipid moiety and said peptide moiety via said coupling moiety is by way of an amide bond between a carbonyl function of said lipid moiety and an amino function of said coupling moiety.

In a preferred embodiment, said linking of said lipid moiety and said peptide moiety via said coupling moiety is by way of an amide bond between a carbonyl function of said lipid moiety and an amino function of said coupling moiety, wherein said coupling moiety is an amino acid linker, preferably consisting of 2-15 amino acids, preferably 2-10 amino acids, and wherein said amino function is the N-terminal amino function of said coupling moiety.

In a preferred embodiment, said linking of said lipid moiety and said peptide moiety via said coupling moiety is by way of an amide bond between a carbonyl function of said lipid moiety and an amino function of said coupling moiety, wherein said coupling moiety is an amino acid linker, preferably consisting of 2-5 amino acids, and wherein said amino function is the N-terminal amino function of said coupling moiety.

In a preferred embodiment, said direct linking of said lipid moiety and said peptide moiety is by way of an amide bond between a carbonyl function of said lipid moiety and an amino function of said peptide moiety, wherein said amino function is the N-terminal amino function of said peptide moiety.

In a preferred embodiment, two Gly residues are included as linker between the lipid moiety, preferably said (R,R)-Pam₂Cys moiety LM-I*1 of the present invention and the start of the coiled-coil heptad repeats, typically and preferably the coiled coil peptide chain segment comprising, preferably consisting of, the sequence IEKKIES (SEQ ID NO:13).

The introduction of an amino acid linker, and preferably a short amino acid linker consisting of two amino acids, preferably glycine, allows during peptide synthesis that after each amino acid coupling, a capping step can be performed with acetic anhydride. This has the practical advantage that after completion of peptide assembly, and coupling of the lipid moieties, preferably the lipid moieties consisting of the formula LM-I*1 [(R,R)-Pam₂Cys moiety] or formula LM-I*2 [(R,R)-Pam₃Cys moiety] to the free N-terminus, the HPLC retention time of the peptide is dramatically altered by lipidation, thus greatly facilitating HPLC purification of the desired lipopeptide building block of the present invention.

In a preferred embodiment, said peptide moiety further comprises a T-helper cell epitope. In a preferred embodiment, said peptide moiety further comprises a T-helper cell epitope, wherein said T-helper cell epitope comprises or preferably consists of a sequence selected from the group consisting of (i) SEQ ID NO:6, SEQ ID NO:63 to SEQ ID NO:87 and (ii) SEQ ID NO:6, SEQ ID NO:63 to SEQ ID NO:87, wherein one, two, or three amino acids are exchanged by other amino acids or are deleted. In a preferred embodiment, said peptide moiety further comprises a T-helper cell epitope, wherein said T-helper cell epitope consists of a sequence selected from the group consisting of (i) SEQ ID NO:6, SEQ ID NO:63 to SEQ ID NO:87 and (ii) SEQ ID NO:6, SEQ ID NO:63 to SEQ ID NO:87, wherein one, two, or three amino acids are exchanged by other amino acids or are deleted. In a preferred embodiment, said peptide moiety further comprises a T-helper cell epitope, wherein said T-helper cell epitope comprises a sequence selected from the group consisting of SEQ ID NO:6, SEQ ID NO:63 to SEQ ID NO:87. In a preferred embodiment, said T-helper cell epitope consists of a sequence selected from the group consisting of SEQ ID NO:6, SEQ ID NO:63 to SEQ ID NO:87.

In one embodiment, said peptide moiety further comprises an amino acid sequence which includes one or more T-helper cell epitopes, and/or strings of polar residues that promote the solubility of the lipopeptide building block in water. Suitable T-helper cell epitopes are known to the skilled person in the art and are described, e.g., in Weber et al., Advanced Drug Delivery Reviews, 2009, 61:11, 965-976; Caro-Aguilar et al., Infect. Immun., 2002, 70:7, 3479-3492; Mishra et al., Immunology, 1993, 79:3, 362-367; Kobayashi et al., Cancer Research, 2000, 60:18, 5228-523; Fraser et al., Vaccine, 2014, 32:24, 2896-2903; Grabowska et al., Int. J. Cancer, 2015, 136:1, 212-224 and WO1998/023635A1. More preferred T-helper cell epitopes included in the peptide moiety are those listed in WO 2015/082501 such TT830-843, TT1064-1079, TT1084-1099, TT947-968, TT1174-1189, DTD271-290, DTD321-340, DTD331-350, DTD351-370, DTD411-430, DTD431-450, TT632-651, CTMOMP36-60, TraT1, TraT2, TraT3, HbcAg5M-69, HbSAg9-33, HA307-319, MA17-31, MVF258-277, MVF288-302, CS.T3, SM Th, PADRE1 and PADRE2 as well as variants thereof in which one, two, or three amino acids are inserted, replaced by other amino acids or deleted.

Preferred T-helper epitopes that can be incorporated into said peptide moiety are any one selected from the group listed in Table 1 below, and variants thereof in which one, two, or three amino acids are replaced by other amino acids or are deleted.

TABLE 1

| T-helper | SEQ ID | |
|---|---|---|
| epitope | NO: | Sequence^{a)} |
| TT830-843 | 63 | QYIKANSKFIGITE |
| TT1064-1079 | 64 | IREDNNITLKLDRCNN |
| TT1084-1099 | 65 | VSIDKFRIFCKANPK |
| TT947-968 | 66 | FNNFTVSFWLRVPKVSASHLET |
| TT1174-1189 | 67 | LKFIIKRYTPNNEIDS |
| DTD271-290 | 68 | PVFAGANYAAWAVNVAQVID |
| DTD321-340 | 69 | VHHNTEEIVAQSIALSSLMV |
| DTD331-350 | 70 | QSIALSSLMVAQAIPLVGEL |
| DTD351-370 | 71 | VDIGFAAYNFVESIINLFQV |
| DTD411-430 | 72 | QGESGHDIKITAENTPLPIA |
| DTD431-450 | 73 | GVLLPTIPGKLDVNKSKTHI |
| TT632-651 | 74 | TIDKISDVSTIVPYIGPALN |
| CTMOMP36-60 | 75 | ALNIWDRFDVFCTLGATTGYLKGNS |
| TraT1 | 76 | GLQGKIADAVKAKG |
| TraT2 | 77 | GLAAGLVGMAADAMVEDVN |
| TraT3 | 78 | STETGNQHHYQTRVVSNANK |
| HbcAg50-69 | 79 | PHHTALRQAILCWGELMTLA |
| HbSAg19-33 | 80 | FFLLTRILTIPQSLD |
| HA307-319 | 81 | PKYVKQNTLKLAT |
| MA17-31 | 82 | YSGPLKAEIAQRLEDV |
| MVF258-277 | 83 | GILESRGIKARITHVDTESY |
| MVF288-302 | 84 | LSEIKGVIVHRLEGV |
| CS.T3 | 6 | IEKKIAKMEKASSVFNVVNS |
| SM Th | 85 | KWFKTNAPNGVDEKIRI |
| PADRE1^{b)} | 86 | aKFVAAWTLKAAa |
| PADRE2^{b)} | 87 | aK-Chx-VAAWTLKAAa |

^{a)}References: SEQ ID NO: 63-67 and 17-20: Eur. J. Immunol. 2001, 31, 3816-3824; SEQ ID NO: 68-74: JID 2000, 181, 1001-1009; SEQ ID NO: 75-78, 83-84 and 85: U.S. 5,759,551; SEQ ID NO: 6: Nature 1988, 336, 778-780; SEQ ID NO: 86-87: Immunity 1994, 1, 751-761.
^{b)},,a“ denotes D-Ala and ,,Chx“ denotes cyclohexylalanine.

Thus, in a further preferred embodiment, said T-helper cell epitope comprises or preferably consists of a sequence selected from the group consisting of (i) SEQ ID NO:1 to SEQ ID NO:26 and (ii) SEQ ID NO:1 to SEQ ID NO:26, wherein one, two, or three amino acids are exchanged by other amino acids or are deleted.

In a preferred embodiment, said peptide moiety further comprises a T-helper cell epitope, wherein said T-helper cell epitope comprises or preferably consists of the amino acid sequence of SEQ ID NO:6. In a preferred embodiment, said peptide moiety further comprises a T-helper cell epitope, wherein said T-helper cell epitope consists of the amino acid sequence of SEQ ID NO:6. In a preferred embodiment, said T-helper cell epitope consists of the sequence of SEQ ID NO:6.

In a further very preferred embodiment, said peptide moiety comprises or preferably consists of (SEQ ID NO:88): GGIEKKIESIEKKIESIEKKIESIEKKIESIEKKIAKMEKASSVFNVVNSKKKC. In a further very preferred embodiment, said peptide moiety consists of (SEQ ID NO:88).

In a further very preferred embodiment, said lipopeptide building block is of the formula LBB-2 or LBB-3, preferably of LBB-2

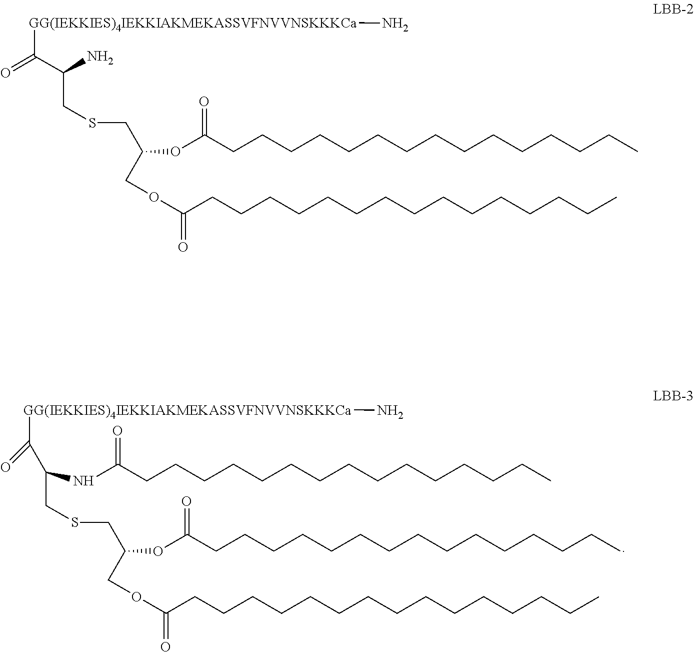

In a further very preferred embodiment, said lipopeptide building block is of the formula LBB-2. In a further very preferred embodiment, said lipopeptide building block is of the formula LBB-3.

In a further aspect, the present invention provides a lipopeptide building block consisting of
(i) a peptide moiety comprising a coiled coil peptide chain segment, and wherein said coiled coil peptide chain segment comprises, preferably consists of, the sequence of (SEQ ID NO:62);
(ii) a lipid moiety comprising, preferably consisting of, the formula LM-I

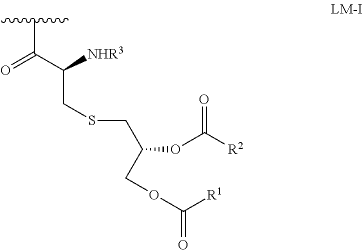

wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁;
and wherein said lipid moiety is linked to said peptide moiety, wherein the wavy line in formula LM-I indicates the linkage site to said peptide moiety, and wherein preferably said lipid moiety is linked to the N-terminus of said peptide moiety.

In a further aspect, the present invention provides a conjugate comprising (a) a lipopeptide building block of the present invention and (b) an antigen, wherein said antigen is connected, directly or via a linker, to said lipopeptide building block.

One or more antigens may be conjugated to the peptide moiety, for example, through one or more of the side chains of amino acids of the peptide moiety, or through the chain terminus of the peptide moiety. The antigen typically carries a functional group suitable for conjugation to a functional group in one of the side chains or the terminus of the peptide moiety. Preferred are antigens recognized by a B-cell receptor, or haptens, in order to elicit antigen-specific antibody-based immune responses.

A variety of coupling or conjugation procedures may be used to attach antigens to the peptide moiety, which will be well known to those knowledgeable in the field. Thus free amino groups in the side chains of amino acids in the peptide moiety of the LBB may be coupled to reactive esters in the antigen (e.g. N-hydroxysuccinimide esters prepared from carboxylic acids); thiols in the peptide moiety may be coupled to maleimide groups in the antigen; azides may be incorporated into the side chains of amino acid residues in the peptide moiety and coupled to antigens containing acetylene groups using copper catalyzed cycloaddition reactions; and other nucleophiles (e.g. hydrazino, hydroxylamino, vic-aminothiol groups) in the peptide may be coupled to electrophiles (e.g. aldehydes, ketones, active esters) in the antigen. It will be obvious that it is possible, in principle, to reverse the positions of the two reactive groups in the peptide chain and antigen in order to achieve selective coupling.

In a further preferred embodiment, said antigen is selected from a peptide, a protein, an epitope mimetic, a carbohydrate or a hapten.

In a further preferred embodiment, said antigen is derived from a source selected from the group consisting of: (a) viruses; (b) bacteria; (c) parasites, preferably protozoan parasites; (d) tumors; (e) self-molecules; (f) non-peptidic hapten molecules; (g) allergens and (h) hormones.

In another preferred embodiment, said antigen is selected from the group consisting of: (1) an antigen suited to induce an immune response against cancer cells; (2) an antigen suited to induce an immune response against infectious diseases; (3) an antigen suited to induce an immune response against allergens; (4) an antigen suited to induce an response against self-molecules; and (5) an antigen suited to induce a response against a drug or a hormone.

In another preferred embodiment, said antigen is a tumor antigen, a self antigen, a polypeptide of a pathogen, an allergen or a hapten.

In another preferred embodiment, said antigen is a tumor antigen, a self antigen, a polypeptide of a pathogen, an allergen or a hapten.

In another preferred embodiment, said antigen is derived from the circumsporozoite (CS) protein of the malaria parasite Plasmodium falciparum. In a further preferred embodiment, said antigen is antigen 43.

In another preferred embodiment, said antigen is a proline rich peptide antigen as described in WO 2015/082501 which is incorporated herein in its entirety by way of reference. In another preferred embodiment, said antigen comprises, preferably consists of, a peptide of SEQ ID NO:27 to 112 as defined in WO 2015/082501 or a peptide of SEQ ID NO:27 to 112 as defined in WO 2015/082501 in which one, two or three amino acids are replaced by other amino acid. In another preferred embodiment, said antigen comprises (i) a peptide of SEQ ID NO:27 to 112 as defined in WO 2015/082501 or (ii) a peptide of SEQ ID NO:27 to 112 as defined in WO 2015/082501 in which one, two or three amino acids are replaced by other amino acid, wherein said antigen consist of 8-80 amino acids, and wherein preferably said antigen consist of 8-60 amino acids, and wherein further preferably said antigen consist of 8-50 amino acids. In another preferred embodiment, said antigen comprises (i) a peptide of SEQ ID NO:27 to 112 as defined in WO 2015/082501 or (ii) a peptide of SEQ ID NO:27 to 112 as defined in WO 2015/082501 in which one, two or three amino acids are replaced by other amino acid, wherein said antigen consist of 8-80 amino acids, and wherein preferably said antigen consist of 8-60 amino acids, and wherein further preferably said antigen consist of 8-50 amino acids. In another preferred embodiment, said antigen comprises (i) a peptide of SEQ ID NO:27 to 83 as defined in WO 2015/082501 or (ii) a peptide of SEQ ID NO:27 to 83 as defined in WO 2015/082501 in which one, two or three amino acids are replaced by other amino acid, wherein said antigen consist of 8-80 amino acids, and wherein preferably said antigen consist of 8-60 amino acids, and wherein further preferably said antigen consist of 8-50 amino acids. In another preferred embodiment, said antigen comprises (i) a peptide of SEQ ID NOs: 27 to 36, 38 to 48, 50 to 55, 61 to 79, 81 to 83 as defined in WO 2015/082501 or (ii) a peptide of SEQ ID NOs: 27 to 36, 38 to 48, 50 to 55, 61 to 79, 81 to 83 as defined in WO 2015/082501 in which one, two or three amino acids are replaced by other amino acid, wherein said antigen consist of 8-80 amino acids, and wherein preferably said antigen consist of 8-60 amino acids, and wherein further preferably said antigen consist of 8-50 amino acids.

In another preferred embodiment, said antigen is a cyclic peptide comprising an amino acid sequence (I), wherein said amino acid sequence (I) comprises, preferably consists of, the amino acid sequence:

(SEQ ID NO: 1)
X1-X2-X3-C4-X5-X6-X7-C8-X9-X10-X11-P12-I13-T14-
N15-D16-Q17-K18-K19-L20-C21-X22-X23-X24-C25-X26-
X27-X28-X29-X30,

wherein
X1, X2, X3, X5, X6, X7, X9, X10, X11, X22, X23, X24, X26, X27, X28 and X29 are independently of each other an amino acid;
C4, C8, C21 and C25 are independently of each other cysteine;
P12 is proline;
113 is isoleucine;
T14 is threonine;
N15 is asparagine;
D16 is aspartic acid;
Q17 is glutamine;
K18 and K19 are independently of each other lysine;
L20 is leucine; and
X30 is an amino acid or a deletion,
wherein said cysteines C4 and C25 form a first disulfide bond and said cysteines C8 and C21 form a second disulfide bond.

The cyclic peptides used in the present invention, when coupled to the inventive lipid building block leading to the inventive conjugates, or further and in particular when incorporated in the inventive synthetic virus-like particles (SVLPs), are able to elicit neutralizing and protective antibodies against the RSV virus, typically, without administration of an adjuvant. Human respiratory syncytial virus (RSV) is a member of the virus family Pneumoviridae and a highly contagious seasonal respiratory virus that infects the lungs and breathing passages. It can spread via droplets sneezed or coughed into the air by an infected person. In particular, RSV is a main cause of lower and upper respiratory tract infections and hospital visits in particular during infancy and childhood but affects older adults and immunocompromised persons as well. Due to high health costs associated with hospitalizations caused by RSV infections, there is a need for prophylaxis of RSV infections. An approach is active immunization by an RSV vaccine. Several different attempts for the generation of such a vaccine have been made (WO 2006/034292, US 2010/0239617, WO 2010/149745, WO2014/144756, WO 2012/048115), but none of the candidates evaluated to date have been approved as safe and effective vaccine for the purpose of preventing RSV infections.

In the used cyclic peptides, the specific disulfide bridges between cysteines C4 and C25 and cysteines C8 and C21 stabilize said peptides by a “helical hairpin” or “helix-tum-helix” conformation. Notably, peptides with no or only one disulfide bridge or with disulfide bridges at different positions have been found to fail to elicit neutralizing antibodies. Moreover, the used cyclic peptides are believed to be well-tolerated vaccines. Immune responses elicited by the cyclic peptides incorporated in SVLPs did not activate VAERD after life RSV infection.

Said cyclic peptides were produced using automated solid-phase peptide synthesis, wherein said disulfide bonds between cysteines C4 and C25 and cysteines C8 and C21 were obtained by oxidative refolding resulting in a beneficial spatial conformation.

In another preferred embodiment, said cyclic peptide has a length of at most 80 amino acids, preferably of at most 60 amino acids, more preferably of at most 40 amino acids, again more preferably of at most 30 amino acids.

In another preferred embodiment, said X11 is selected from norleucine, 6-hydroxy-norleucine, norvaline, 5-oxo-norleucine, 2-aminoheptanoic acid, methionine, ethionine, hydroxy-methionine, s-oxymethionine, methionine sulfone, or methionine sulfoxide, wherein preferably X11 is norleucine.

In another preferred embodiment, said X23 is selected from asparagine, beta-hydroxyasparagine, 2,5-diamino-4-hydroxy-5-oxopentanoic acid, glutamine, glutamine hydroxamate, 3-methyl-glutamine, n-methyl-asparagine, n5-methyl-glutamine, cysteine-s-acetamide; serine, homoserine, allo-threonine, 3,3-dihydroxy-alanine, 2-amino-5-hydroxypentanoic acid, 4-hydroxy-l-threonine, threonine, hydroxynorvaline, 6-hydroxy-l-norleucine or glycine; wherein preferably X23 is asparagine, serine, glutamine or glycine. In a further preferred embodiment, said X23 is serine. In a further preferred embodiment, said X23 is glutamine. In a further preferred embodiment, said X23 is glycine. In a further preferred embodiment, said X23 is asparagine.

In another preferred embodiment, said X24 is selected from asparagine, beta-hydroxyasparagine, 2,5-diamino-4-hydroxy-5-oxopentanoic acid, glutamine, glutamine hydroxamate, 3-methyl-glutamine, n-methyl-asparagine, n5-methyl-glutamine, cysteine-s-acetamide; lysine, 2,4-diaminobutyric acid, 2,3-diaminopropanoic acid, 2,8-diaminooctanoic acid, ornithine, amino-adipic acid, thialysine; aspartic acid, 2-amino-6-oxopimelic acid, 3-methyl-aspartic acid, 1-2-amino-6-methylene-pimelic acid, 4-hydroxy-glutamic-acid, 6-carboxylysine, beta-hydroxyaspartic acid, 3,3-dimethyl aspartic acid, 2-amino-propanedioic acid, glutamate, 5-o-methyl-glutamic acid, (3r)-3-methyl-l-glutamic acid, (3s)-3-methyl-l-glutamic acid, 2s,4r-4-methylglutamate 2-aminoadipic acid, serine, homoserine, allo-threonine, 3,3-dihydroxy-alanine, 2-amino-5-hydroxypentanoic acid, 4-hydroxy-l-threonine, threonine, hydroxynorvaline, 6-hydroxy-l-norleucine or glycine. In another preferred embodiment, X24 is selected from asparagine, lysine, ornithine, 2,4-diaminobutyric acid (Dab), glutamine, glycine or serine. In another preferred embodiment, X24 is glycine, glutamine or serine. In another preferred embodiment, X24 is serine. In another preferred embodiment, X24 is glutamine. In another preferred embodiment, X24 is glycine.

In another preferred embodiment, X11 is norleucine and X24 is selected from asparagine, lysine, ornithine, 2,4-diaminobutyric acid (Dab), glutamine, glycine or serine.

In another preferred embodiment, X11 is norleucine and X24 is serine. In another preferred embodiment, X11 is norleucine and X24 is glutamine. In another preferred embodiment, X11 is norleucine and X24 is asparagine. In another preferred embodiment, X11 is norleucine and X24 is aspartic acid. In another preferred embodiment, X11 is norleucine and X24 is asparagine. In another preferred embodiment, X11 is norleucine and X24 is ornithine. In another preferred embodiment, X11 is norleucine and X24 is 2,4-diaminobutyric acid. In another preferred embodiment, X11 is norleucine and X24 is lysine.

In another preferred embodiment, the C-terminal amino acid of said amino acid sequence (I) is selected from alanine, leucine, valine, norleucine, norvaline, isoleucine, homoleucine, vinylglycine, 2-aminobutyric acid, 2-allylglycine, alloleucine, alloisoleucine, 2-aminoheptanoic acid, serine, glutamine or glycine.

In another preferred embodiment, X11 is norleucine, X24 is selected from lysine, 2,4-diaminobutyric acid, aspartic acid, asparagine, serine, glutamine or glycine, and said C-terminal amino acid of said amino acid sequence (I) is selected from alanine, leucine, valine, norleucine, norvaline, isoleucine, homoleucine, vinylglycine, 2-aminobutyric acid, 2-allylglycine, alloleucine, alloisoleucine, or 2-aminoheptanoic acid. Preferably, said C-terminal amino acid of said amino acid sequence (I) is selected from alanine, leucine, valine, norleucine, norvaline, isoleucine, homoleucine, vinylglycine, 2-aminobutyric acid, 2-allylglycine, alloleucine, alloisoleucine, or 2-aminoheptanoic acid, further preferably or alternatively preferably, said C-terminal amino acid of said amino acid sequence (I) is a D-stereoisomer.

In another preferred embodiment, said C-terminal amino acid of said amino acid sequence (I) is selected from a D-stereoisomer of alanine, leucine, valine, norleucine, norvaline, isoleucine, homoleucine, vinylglycine, 2-aminobutyric acid, 2-allylglycine, alloleucine, alloisoleucine, or 2-aminoheptanoic acid. In another preferred embodiment, said C-terminal amino acid of said amino acid sequence (I) is alanine, preferably D-alanine.

In another preferred embodiment, said X1 is a polar or hydrophobic amino acid. In another preferred embodiment, said X1 is selected from asparagine, beta-hydroxyasparagine, 2,5-diamino-4-hydroxy-5-oxopentanoic acid, glutamine, glutamine hydroxamate, 3-methyl-l-glutamine, n-methyl-asparagine, n5-methyl-glutamine, cysteine-s-acetamide; leucine, alloleucine, alloisoleucine, homoleucine, isoleucine, 2-aminobutyric acid, norleucine, norvaline, valine; serine, homoserine, allo-thonine, 3,3-dihydroxy-alanine, 2-amino-5-hydroxypentanoic acid, 4-hydroxy-l-threonine, threonine, hydroxynorvaline, 6-hydroxy-l-norleucine; or glycine. In another preferred embodiment, said X1 is selected from asparagine, glutamine, leucine, serine, or glycine. In another preferred embodiment, said X1 is glycine. In another preferred embodiment, said X1 is glutamine. In another preferred embodiment, said X1 is serine.

In another preferred embodiment, said X1, X23 and X24 are each independently selected from the group consisting of ornithine, aspartic acid, lysine, asparagine, 2,4-diaminobutyric acid (Dab), glutamine, leucine, serine, and glycine.

In another preferred embodiment, said X1, X23 and X24 are each independently selected from the group consisting of asparagine, 2,4-diaminobutyric acid (Dab), glutamine, leucine, serine, and glycine. In another preferred embodiment, said X1 is selected from glutamine, serine, or glycine; said X23 is serine; and said X24 is glutamine or serine.

In another preferred embodiment, said X2, X6 and X22 are independently of each other a polar amino acid. Preferably, X2, X6 and X22 are independently of each other selected of 2-amino-5-hydroxypentanoic acid, allo-threonine, 4-chloro-threonine, 3,3-dihydroxy-alanine, 4-hydroxy-L-isoleucine, (2s,3r)-2-amino-3-hydroxy-4-methylpentanoic acid, beta-hydroxy-leucine, homoserine, 3-hydroxy-l-valine, 4,5-dihydroxy-isoleucine, 6-hydroxy-l-norleucine, s-(2-hydroxyethyl)-1-cysteine, phosphoserine, serine, 4-hydroxy-l-threonine, threonine, phosphothreonine or hydroxynorvaline. In another preferred embodiment, X2, X6 and X22 are serine.

In another preferred embodiment, said X3 is an amino acid having an acidic or negatively charged side chain at a physiological pH (about pH 7). Preferably, X3 is selected of glutamate, 5-o-methyl-glutamic acid, (3r)-3-methyl-l-glutamic acid, (3s)-3-methyl-l-glutamic acid, 2s,4r-4-methylglutamate, 4-hydroxy-glutamic-acid, 2-aminoadipic acid, 1-2-amino-6-methylene-pimelic acid, 2-amino-6-oxopimelic acid; 3-methyl-aspartic acid, 6-carboxylysine, aspartic acid, beta-hydroxyaspartic acid, 3,3-dimethyl aspartic acid, or 2-amino-propanedioic acid. More preferably, X3 is selected of glutamate, 5-o-methyl-glutamic acid, (3r)-3-methyl-l-glutamic acid, (3s)-3-methyl-l-glutamic acid, 2s,4r-4-methylglutamate, 4-hydroxy-glutamic-acid, 2-aminoadipic acid, 1-2-amino-6-methylene-pimelic acid or 2-amino-6-oxopimelic acid. In another again more preferred embodiment, X3 is glutamate.

In another preferred embodiment, said X5 and X7 are independently of each other a hydrophobic amino acid. Preferably, X5 and X7 are independently of each other selected of leucine, alloleucine, alloisoleucine, homoleucine, isoleucine, 2-aminobutyric acid, norleucine, norvaline or valine. More preferably, X5 or X7 is leucine. In another more again preferred embodiment, X5 and X7 are leucine.

In another preferred embodiment, said X9 and X23 are independently of each other a polar amino acid. Preferably, X9 and X23 are independently of each other selected of asparagine, glutamine, serine or glycine. In another more preferred embodiment, X9 or X23 is independently selected from asparagine, glutamine, serine or glycine. In another again more preferred embodiment, X9 and X23 are both independently selected from asparagine, glutamine, serine or glycine. In another again more preferred embodiment, X9 and X23 are asparagine.

In another preferred embodiment, said X10 is an amino acid having an acidic or negatively charged side chain at a physiological pH (about pH 7). Preferably, X10 is selected of 3-methyl-aspartic acid, 6-carboxylysine, aspartic acid, beta-hydroxyaspartic acid, 3,3-dimethyl aspartic acid, or 2-amino-propanedioic acid. In another again more preferred embodiment, X10 is aspartic acid.

In another preferred embodiment, said X26 is a hydrophobic or polar amino acid. Preferably, X26 is selected of leucine, alloleucine, alloisoleucine, homoleucine, isoleucine, 2-aminobutyric acid, norleucine, norvaline, valine; 2,5-diamino-4-hydroxy-5-oxopentanoic acid, glutamine hydroxamate, 3-methyl-l-glutamine, n5-methyl-glutamine, asparagine, 2,5-diamino-4-hydroxy-5-oxopentanoic acid, or n-methyl-asparagine. More preferably, X26 is leucine or glutamine.

In another preferred embodiment, said X27 is a polar or hydrophobic amino acid or an amino acid having an acidic or negatively charged side chain at a physiological pH (about pH 7). Preferably, X27 is serine, isoleucine, or lysine.

In another preferred embodiment, said X28 is a polar or hydrophobic amino acid. Preferably, X28 is valine or serine.

In another preferred embodiment, said X29 is a hydrophobic amino acid or an amino acid having a negatively charged side chain at physiological pH (about pH 7). Preferably, X29 is D- or L-alanine or D- or L-arginine. In another more preferred embodiment, X29 is D-alanine or D-arginine.

In another preferred embodiment, said X30 is a deletion or a hydrophobic or polar D- or L-amino acid, preferably X30 is a hydrophobic or polar amino acid D-amino acid. Preferably, X30 is a deletion or X30 is D- or L-glutamine or D- or L-alanine. In another more preferred embodiment, X30 is D-glutamine or D-alanine. In another again more preferred embodiment, X30 is D-alanine. In a preferred embodiment, X30 is a deletion and X29 is alanine, preferably D-alanine. In another preferred embodiment, X30 is alanine, preferably D-alanine and X29 is arginine.

In another preferred embodiment, X1 is asparagine or leucine, X2 is serine, and X3 is glutamate. In another preferred embodiment, X1 is serine, glycine, glutamine, asparagine or leucine, X2 is serine, and X3 is glutamate. In another preferred embodiment, X5 is leucine, X6 is serine, and X7 is leucine. In another preferred embodiment, X9 is asparagine, X10 is aspartic acid and X11 is norleucine or methionine, preferably X11 is norleucine.

In another preferred embodiment, X22 is serine, X23 is asparagine, and X24 is serine, glycine, glutamine, Dab (2,4-diaminobutyric acid), asparagine or lysine, preferably X24 is Dab (2,4-diaminobutyric acid) or lysine. In another preferred embodiment, X26 is glutamine or leucine, X27 is serine, lysine or isoleucine, X28 is valine, X29 is D- or L-arginine or D- or L-alanine, preferably X29 is D- or L-alanine, more preferably X29 is D-alanine, and X30 is a deletion, D- or L-alanine or D- or L-glutamine, preferably X30 is D- or L-alanine, more preferably X30 is D-alanine.

In another preferred embodiment, X1 is asparagine or leucine, X2 is serine, X3 is glutamate, X5 is leucine, X6 is serine, and X7 is leucine, and preferably X9 is asparagine, X10 is aspartic acid and X11 is norleucine or methionine, and further preferably X11 is norleucine. In another preferred embodiment, X1 is asparagine or leucine, X2 is serine, X3 is glutamate, X22 is serine, X23 is asparagine, and X24 is Dab (2,4-diaminobutyric acid), asparagine or lysine, preferably X24 is Dab (2,4-diaminobutyric acid) or lysine, and preferably X26 is glutamine or leucine, X27 is serine, lysine or isoleucine, X28 is valine, X29 is D- or L-arginine or D- or L-alanine, further preferably X29 is D- or L-alanine, more preferably X29 is D-alanine, and X30 is a deletion, D- or L-alanine or D- or L-glutamine, further preferably X30 is D- or L-alanine, more preferably X30 is D-alanine.

In another preferred embodiment, X1 is asparagine or leucine, X2 is serine, X3 is glutamate, X5 is leucine, X6 is serine, and X7 is leucine. In another preferred embodiment, X1 is asparagine or leucine, X2 is serine, X3 is glutamate, X9 is asparagine, X10 is aspartic acid and X11 is norleucine or methionine, preferably X11 is norleucine. In another preferred embodiment, X1 is asparagine or leucine, X2 is serine, X3 is glutamate, X22 is serine, X23 is asparagine, and X24 is Dab (2,4-diaminobutyric acid), asparagine or lysine, preferably X24 is Dab (2,4-diaminobutyric acid) or lysine. In another preferred embodiment, X1 is asparagine or leucine, X2 is serine, X3 is glutamate, X26 is glutamine or leucine, X27 is serine, lysine or isoleucine, X28 is valine, X29 is D- or L-arginine or D- or L-alanine, preferably X29 is D- or L-alanine, more preferably X29 is D-alanine, and X30 is a deletion, D- or L-alanine or D- or L-glutamine, preferably X30 is D- or L-alanine, more preferably X30 is D-alanine.

In a preferred embodiment, said X2, X3, X5, X6, X7, X9, X10, X11, X22, X23, X24, X26, X27, X28 and X29 are independently of each other L-amino acids. In a preferred embodiment proline P12, isoleucine 113, threonine T14, asparagine N15, aspartic acid D16, glutamine Q17, lysines K18 and K19, and leucine L20 are independently of each other L-amino acids. In a preferred embodiment, C4, C8, C21 and C25 are independently of each other D-cysteine or L-cysteine, preferably L-cysteine.

In a preferred embodiment, said cyclic peptide consists of said amino acid sequence (I). In another preferred embodiment, said amino acid sequence (I) consists of said amino acid sequence of SEQ ID NO:1.

In another very preferred embodiment, said amino acid sequence of SEQ ID NO:1 is selected from any one of SEQ ID NO:2-5, 21-36, or 39-57.

In another very preferred embodiment, said amino acid sequence of SEQ ID NO:1 is selected from any one of SEQ ID NO:39-57.

In another very preferred embodiment, said amino acid sequence of SEQ ID NO:1 is selected from any one of SEQ ID NO:2-5, 21-36.

In another very preferred embodiment, said amino acid sequence (I) is selected from any one of (SEQ ID NO:2), (SEQ ID NO:3), (SEQ ID NO:4), (SEQ ID NO:5), (SEQ ID NO:21), (SEQ ID NO:22), (SEQ ID NO:23), (SEQ ID NO:24), (SEQ ID NO:25), (SEQ ID NO:26), (SEQ ID NO:27), (SEQ ID NO:28), (SEQ ID NO:29), (SEQ ID NO:30), (SEQ ID NO:31), (SEQ ID NO:32), (SEQ ID NO:33), (SEQ ID NO:34), (SEQ ID NO:35), or (SEQ ID NO:36).

In another very preferred embodiment, said amino acid sequence of SEQ ID NO:1 is any one of SEQ ID NO:2, 3, 21, 22, or 23.

In another very preferred embodiment, said amino acid sequence of SEQ ID NO:1 is an amino acid selected from SEQ ID NO:2 or SEQ ID NO:3.

In a certain embodiment, said amino acid sequence (I) of said cyclic peptide comprises (i) an N-terminus selected from a free amino group or an acetylated N-terminus, and/or (ii) a C-terminus selected from a free carboxyl group or an amidated C-terminus.

In another preferred embodiment, said cyclic peptide further comprises a linker, wherein said linker is attached to said amino acid sequence (I), and wherein said linker comprises (i) at least one attachment moiety, (ii) at least one spacer moiety, (iii) at least one, preferably one, linking moiety, or (iv) any combination of (i), (ii) and (iii).

In another preferred embodiment, said at least one attachment moiety comprises or preferably consists of —O—NH₂, —O—NH— (an aminooxy moiety), —C(O)—CH₂—O—NH₂, —C(O)—CH₂—O—NH— (aminooxy acetyl moiety), —NH—NH₂, —NH—NH— (hydrazine moiety), -E(O)—NH—NH₂, or -E(O)—NH—NH— (hydrazide moiety), wherein E is C, S(O) or P. In a further preferred embodiment, said attachment moiety comprises or preferably consists of an —O—NH₂, —O—NH— (an aminooxy moiety), —C(O)—CH₂—O—NH₂, —C(O)—CH₂—O—NH— (aminooxy acetyl moiety), —NH—NH₂, —NH—NH— (hydrazine moiety), or (—C(O)—NH—NH₂, —C(O)—NH—NH-(carbohydrazide moiety). In another further preferred embodiment, said attachment moiety comprises or preferably consists of —O—NH₂ or —O—NH— (an aminooxy moiety). In another preferred embodiment, said at least one spacer moiety comprises or preferably consists of NH₂—CH₂—CH₂—(O—CH₂—CH₂)ₙ—C(O)— or —NH—CH₂—CH₂—(O—CH₂—CH₂)ₙ—C(O)—, wherein n is an integer of 1 to 45, preferably 2 to 20, more preferably 6 to 8; or NH₂—(CH₂)ₘ—C(O)— or —NH—(CH₂)ₘ—C(O)—, wherein m is an integer of 2 to 45, preferably 2 to 20, more preferably 2 to 6. In another preferred embodiment, said at least one linking moiety is capable of cross-linking the cyclic peptide with a second peptide. Linking moieties capable of cross-linking a cyclic peptide with a second peptide are well known in the art. In one embodiment of the invention, said linking moiety capable of cross-linking the cyclic peptide with a second peptide comprises or consists of an aldehyde moiety, such as a glutaraldehyde moiety, octanedialdehyde moiety, dialdehyde moiety, succinaldehyde moiety; carbodiimide moiety, such as 1-ethyl-3-(3-dimethylaminopropyl)carbodiimide hydrochloride moiety; glyoxol moiety; N-hydroxy-sulphosuccinimidyl moiety, such as N-hydroxy-sulphosuccinimidyl moiety; a cationic linking moiety; polyethyleneglycol moiety; benzoyl benzoic acid moiety. Further suitable linking moieties are listed in the Pierce Catalog and Handbook, Pierce Chemical Company, Rockford (1997); Bioconjugate Techniques, Greg T. Hermanson, Pierce Biotechnology, Thermo Fisher Scientific, Rockford (2013); and are described in EP 1321466 A1, DE 19821859 A1, U.S. Pat. Nos. 6,875,737, 5,456,911, 5,612,036, 5,965,532, WO 2001004135, WO 2001070685, US 20140302001 A1, U.S. Pat. No. 6,800,728, US 20140171619 A1, U.S. Pat. No. 8,168,190, WO 2012/166594 A1 and WO 2015/082501.

In one embodiment, said linker is at least one, preferably exactly one attachment moiety. In another embodiment, said linker is at least one, preferably exactly one, two or three spacer moieties, wherein in case of more than one spacer moiety, they are preferably linked together, either directly or via an interconnecting group. In another embodiment, said linker is at least one, preferably exactly one spacer moiety. In another embodiment, said linker is at least one, preferably exactly one linking moiety.

In another embodiment, said linker comprises at least one attachment moiety and at least one, preferably exactly one spacer moiety, wherein the at least one attachment moiety is attached to the N-terminus of said amino acid sequence (I), and the at least one spacer moiety is attached to the attachment moiety. In another embodiment, said linker comprises at least one attachment moiety and at least one, preferably exactly one spacer moiety wherein the at least one spacer moiety is attached to the N-terminus of said amino acid sequence (I), and the at least one attachment moiety is attached to the spacer moiety.

In one embodiment, said at least one linker comprises at least one attachment moiety and at least one spacer moiety and at least one linking moiety, wherein the at least one linking moiety or the at least one spacer moiety is attached to the N-terminus of said amino acid sequence (I). In another embodiment, the at least one attachment moiety is attached to the N-terminus of said amino acid sequence (I), the at least one spacer moiety is attached to the attachment moiety and the at least one linking moiety is attached to the spacer moiety. In another embodiment, the at least one spacer moiety is attached to the N-terminus of the amino acid sequence (I), the at least one attachment moiety is attached to the spacer moiety and the at least one linking moiety is attached to the attachment moiety.

In a preferred embodiment, the linker is attached to the amino acid sequence (I) typically and preferably via an amide bond to the N-terminus of said amino acid sequence (I) or to a free amino group of a side chain of an amino acid of said amino acid sequence (I), preferably to the N-terminus of said amino acid sequence (I). In a preferred embodiment, said linking moiety is capable of cross-linking said cyclic peptide with a thiol group of a second peptide. In a preferred embodiment, said linking moiety comprises a maleimide moiety.

In a preferred embodiment, said linker is attached to an amino group included in said amino acid sequence (I), wherein preferably said linker is attached to a free amino group of (i) the N-terminus of said amino acid sequence (I), or (ii) a side chain of an amino acid of said amino acid sequence (I). Preferably, the linker is attached to said amino group included in said amino acid sequence (I) by an amide bond. Said side chain is preferably of the amino acid lysine. In a preferred embodiment, X24 is lysine and said linker is attached to the free amino group of the side chain of X24.

In a very preferred embodiment, said linker is selected from the following formulas:

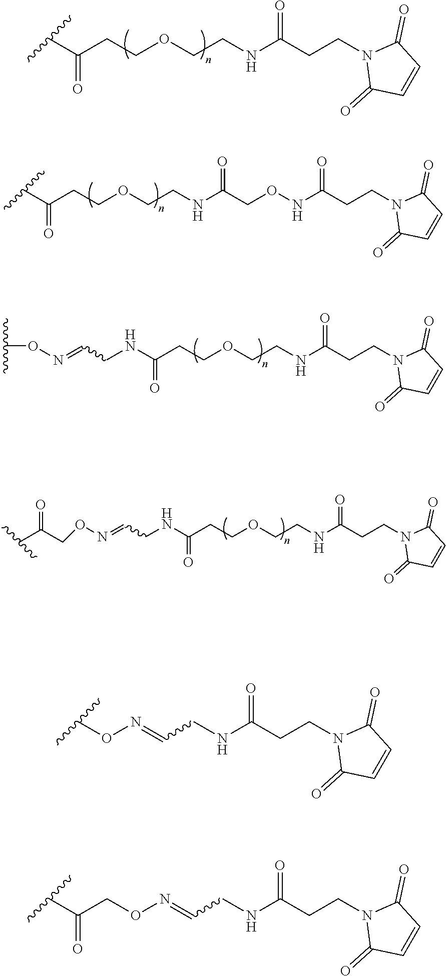

wherein n is an integer of 1 to 45, preferably 6 to 8, and the wavy line indicates the attachment site to said amino acid sequence (I). Further very preferred, said n is 6.

In case the compounds of the present invention such as the conjugate, and hereby including said linker, comprise one or more double bonds, said double bonds can be of either the (E)- or (Z)-configuration, or mixtures thereof in any ratio. The same applies for the preferred linker comprising an oxime moiety. Thus, the preferred linker comprising an oxime moiety thus may include either said linker with said oxime moiety in its syn-configuration (and thus as syn-isomer), said linker with said oxime moiety in its anti-configuration (and thus as anti-isomer) and mixtures thereof in any ratio. Within the chemical formulas presented herein for said double bond or said oxime moiety, this is typically and preferably represented by a wavy line.

In a further very preferred embodiment, said linker is selected from the following formulas:

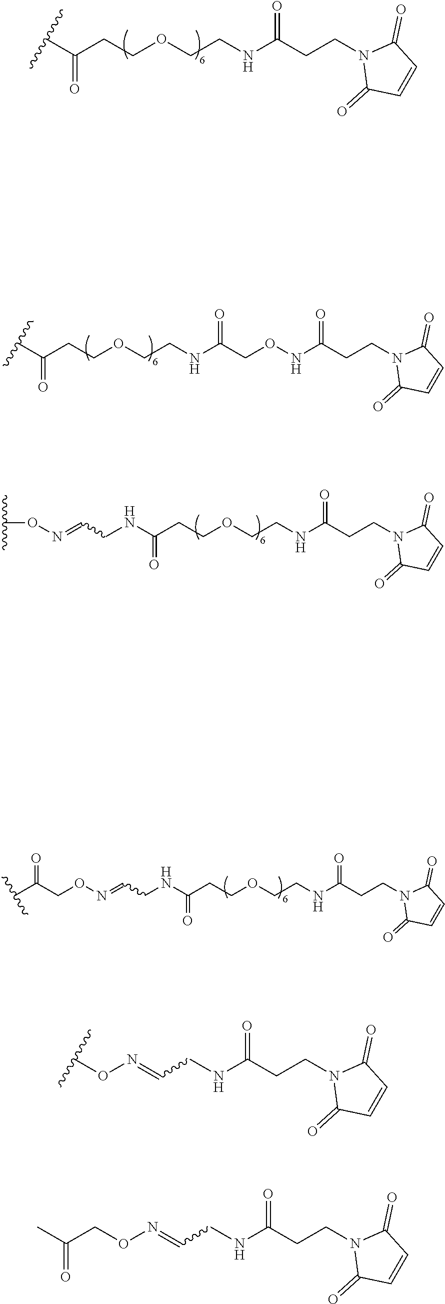

wherein the wavy line indicates the attachment site to said amino acid sequence (I).

In a further very preferred embodiment, said cyclic peptide comprises, preferably is, a formula selected from any one of following formulas:

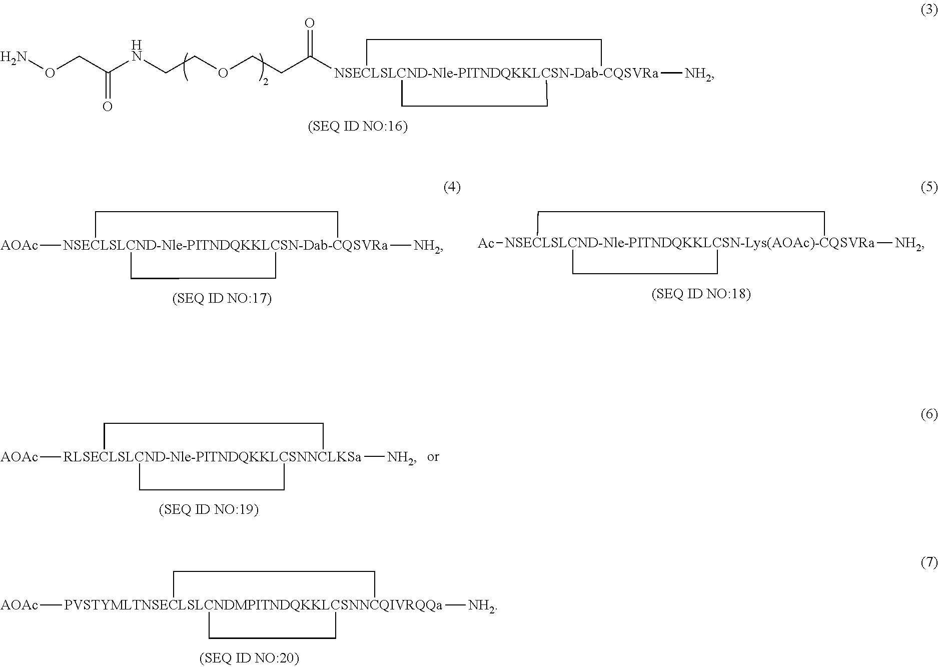

In a further very preferred embodiment, said cyclic peptide comprises, preferably is, a formula selected from any one of formulas (3) (SEQ ID NO:16), formula (4) (SEQ ID NO:17), formula (5) (SEQ ID NO:18), formula (6) (SEQ ID NO:19), formula (7) (SEQ ID NO:20), and any of formulas (19) to (37) as depicted in the Example Section.

In a further very preferred embodiment, said cyclic peptide comprises, preferably is, formula (3) (SEQ ID NO:16). In a further very preferred embodiment, said cyclic peptide comprises, preferably is, formula (4) (SEQ ID NO:17). In a further very preferred embodiment, said cyclic peptide comprises, preferably is, formula (5) (SEQ ID NO:18). In a further very preferred embodiment, said cyclic peptide comprises, preferably is, formula (6) (SEQ ID NO:19). In a further very preferred embodiment, said cyclic peptide comprises, preferably is, formula (7) (SEQ ID NO:20).

Linking and conjugation procedures that may be used to attach the cyclic peptide to the lipopeptide building block are well known to those skilled in the art (see for example Hermanson, G. T, Bioconjugate Techniques, 2nd edition, Academic Press, 2008). Any method used for linking and conjugating peptides or other antigens to an antigen delivery system, such as carrier protein, polymer, dendrimer, nanoparticle or virus-like particle, can be used to link said cyclic peptide to said lipopeptide building block. Free amino groups in the side chains of amino acids in the peptide moiety of the lipopeptide building block may be coupled to reactive esters in the cyclic peptide or the linker (e.g. N-hydroxysuccinimide esters prepared from carboxylic acids); thiols in the peptide moiety may be coupled to maleimide groups in the linker; azides may be incorporated into the side chains of amino acid residues in the peptide moiety and coupled to the cyclic peptide or linker containing acetylene groups using copper catalyzed cycloaddition reactions; and other nucleophiles (e.g. hydrazino, hydroxyl amino, vic-amino thiol groups) in the peptide moiety may be coupled to electrophiles (e.g. aldehydes, ketones. active esters) in the cyclic peptide or linker. Further, the reversal of the positions of the two reactive groups in the peptide moiety and cyclic peptide or linker to achieve selective coupling is further envisaged.

All embodiments and preferred and very preferred embodiments of the lipid building block, the conjugate, and all of its components including antigens and linker described herein are applicable to all aspects of the present invention, even though not all embodiments and preferred and very preferred embodiments are not necessarily again repeated and reiterated.

In a further very preferred embodiment, said conjugate is selected from any one of the formula

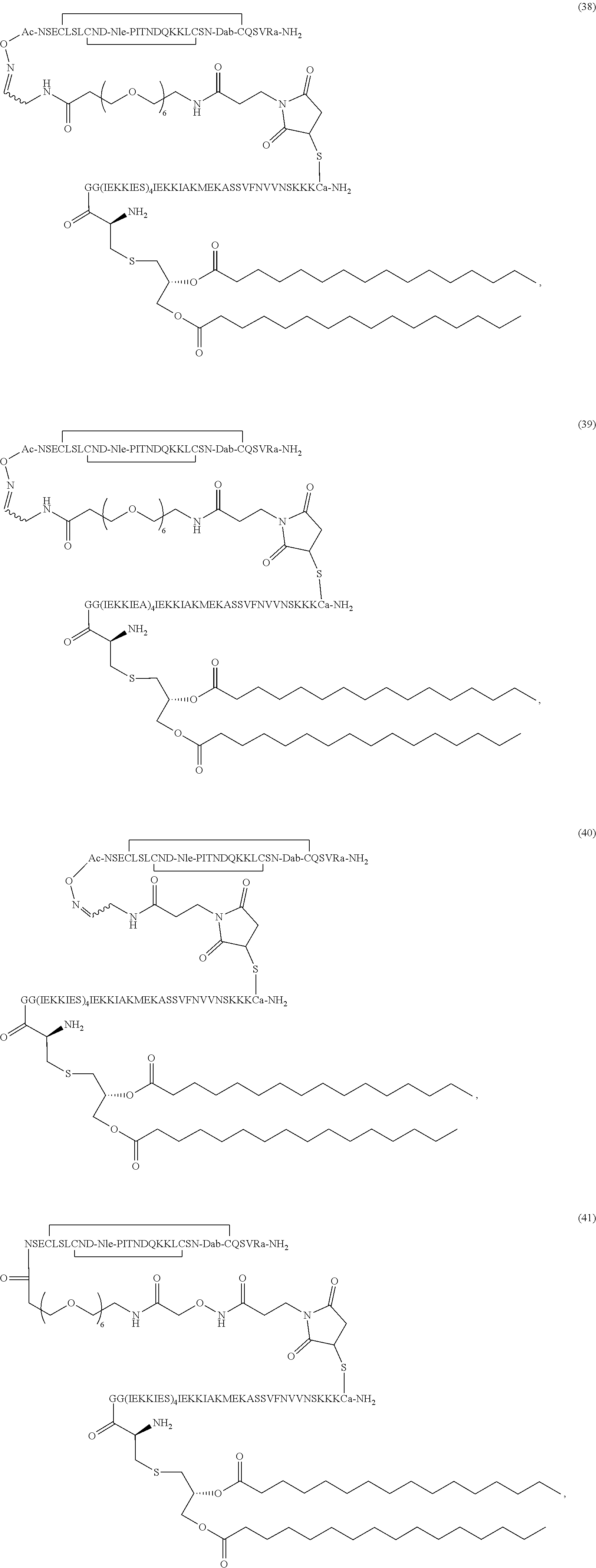

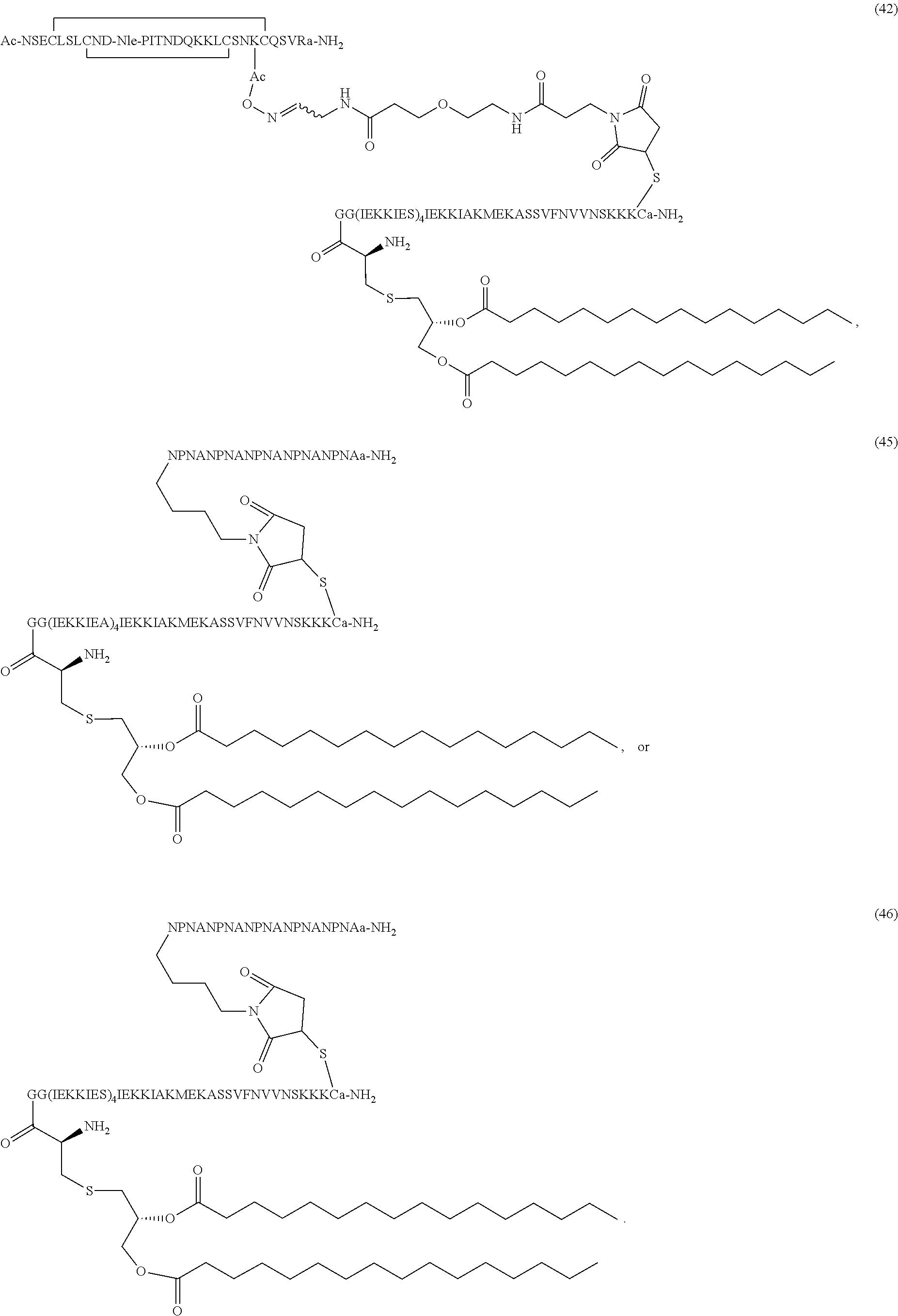

In a further very preferred embodiment, said conjugate is (38).

In a further very preferred embodiment, said conjugate is (39).

In a further very preferred embodiment, said conjugate is (40).

In a further very preferred embodiment, said conjugate is (41).

In a further very preferred embodiment, said conjugate is (42).

In a further very preferred embodiment, said conjugate is (45).

In a further very preferred embodiment, said conjugate is (46),

In another aspect, the present invention provides for a bundle of conjugates comprising 2, 3, 4, 5, 6 or 7 of the inventive conjugate. In another aspect, the present invention provides for a bundle of conjugates comprising 2, 3, 4 or 5 of the inventive conjugate. In another very preferred aspect, the present invention provides for a bundle of conjugates comprising 3 of the inventive conjugate.

In another aspect, the present invention provides for a bundle of conjugates comprising 2, 3, 4, 5, 6 or 7 of the inventive conjugate, wherein said conjugate is selected from any one of the formula (38), (39), (40), (41), (42), (45) or (46), wherein preferably said conjugate is selected from any one of the formula (38), (40), (41), (42) or (46), and wherein further preferably said conjugate is formula (38) or (46) preferably (38).

In another very preferred aspect, the present invention provides for a bundle of conjugates comprising 3, preferably exactly 3, of the inventive conjugate, wherein said conjugate is selected from any one of the formula (38), (39), (40), (41), (42), (45) or (46), wherein preferably said conjugate is selected from any one of the formula (38), (40), (41), (42) or (46), and wherein further preferably said conjugate is formula (38) or (46) preferably (38).

According to a preferred embodiment, in said bundle, the coiled coil peptide chain segments of said peptide moieties comprised by said conjugates are coiled together, preferably said coiled coil peptide chain segments are helically coiled together, more preferably said coiled coil peptide chain segments are alpha-helically coiled together. In a preferred embodiment, said coiled coil peptide chain segments of said peptide moieties are coiled together left-handed or right-handed. According a preferred embodiment, in said bundle, said coiled coil peptide chain segments of said peptide moieties form an alpha-helical left-handed coil.

In a preferred embodiment, said coiled coil peptide chain segments have a parallel orientation, i.e. they run in the same direction; or they have an anti-parallel orientation, i.e. they run in directions opposite to each other; wherein the first option is preferred. The term “direction” is based on the direction of a peptide chain having on one side an N-terminus and on the other side a C-terminus. In a preferred embodiment of said inventive bundle, said coiled coil peptide chain segments of said peptide moieties form a left-handed alpha-helical coiled coil, wherein the coiled coil peptide chain segments have a parallel orientation in said coiled coil. Preferably, said bundle comprises 2 to 7 (e.g. dimer, trimer, tetramer, pentamer, hexamer or heptamer), more preferably 2, 3, 4 or 5, again more preferably 3 helically twisted coiled coil peptide chain segments, having a parallel orientation in said coiled coil.

In another aspect, the present invention provides for a synthetic virus-like particle comprising at least one bundle of conjugates of the present invention.

In another aspect, the present invention provides for a synthetic virus-like particle comprising at least one bundle of conjugates of the present invention, wherein said conjugate is selected from any one of the formula (38), (39), (40), (41), (42), (45) or (46), wherein preferably said conjugate is selected from any one of the formula (38), (40), (41), (42) or (46), and wherein further preferably said conjugate is formula (38) or (46) preferably (38).

The invention also relates to a method of preparing the synthetic virus-like particles of the invention. Synthetic virus-like particles (SVLP) may be produced by a self-assembly process, e.g. in aqueous solution. This method may involve dissolving the lipopeptide building block in a suitable carrier, preferably an aqueous buffer system (e.g. buffered saline or unbuffered saline). The solvent may be removed after preparation of the synthetic virus-like particles, for example by lyophilization or spray drying. Conjugates including the specific combination of the cyclic peptide of the invention and the lipopeptide building block of the invention self-assemble to bundles and further to synthetic virus-like particles (SVLPs).

In another aspect, the present invention provides for a pharmaceutical composition comprising an immunologically effective amount of the conjugate of the present invention or the synthetic virus like particle of the present invention, together with a pharmaceutically acceptable diluent, carrier or excipient, wherein preferably said pharmaceutical composition is a vaccine.

As used herein, the term “effective amount” refers to an amount necessary or sufficient to realize a desired biologic effect. Preferably, the term “effective amount” refers to an amount of the antigen of the present invention, the conjugate of the present invention or the synthetic virus like particle of the present invention that (i) treats or prevents the particular disease, medical condition, or disorder, (ii) attenuates, ameliorates, or eliminates one or more symptoms of the particular disease, medical condition, or disorder, or (iii) prevents or delays the onset of one or more symptoms of the particular disease, medical condition, or disorder described herein. An immunogenically effective amount, as herein understood, is an amount that is capable of modulating, preferably enhancing the response of the immune system of a subject to an antigen or pathogen.

The invention further relates to the conjugate or the synthetic virus like particle of the invention for use as a vaccine. A vaccine, as used herein, is a pharmaceutical composition that is used to modulate, preferably to stimulate the response of the body's immune system to a particular antigen or pathogen. In a preferred embodiment, the pharmaceutical composition or preferably the vaccine is used for preventing or reducing the risk of an RSV infection in a subject, preferably a human, more preferably a child or elderly people.

In again another aspect, the present invention provides for the conjugate of the present invention or the synthetic virus like particle of the present invention for use as a medicament, preferably for use in a method for preventing a disease or for reducing the risk of a disease, wherein further preferably said disease is an infectious disease, a cancer or an allergy, and again more preferably wherein said disease is an respiratory syncytial virus (RSV) infection.

In again another aspect, the present invention provides for the conjugate of the present invention or the synthetic virus like particle of the present invention for use in a method for preventing a disease or for reducing the risk of a disease or for treating a disease, wherein further preferably said disease is an infectious disease, a cancer or an allergy, and again more preferably wherein said disease is an respiratory syncytial virus (RSV) infection. In again another aspect, the present invention provides for the conjugate of the present invention or the synthetic virus like particle of the present invention for use in a method for preventing of an infectious disease or reducing the risk of an infectious disease, preferably for use in a method for preventing or reducing the risk of an infectious disease associated with or caused by a respiratory syncytial virus. As used herein, the term treating refers to therapy and to a therapeutic treatment.

The present invention provides for the conjugate of the invention, the synthetic virus like particle of the invention, or the pharmaceutical composition of the invention for use as a medicament, preferably for use in a method for preventing an infectious disease or for reducing the risk of an infectious disease, more preferably for use in a method for preventing or reducing the risk of an infectious disease associated with or caused by a respiratory syncytial virus (RSV). The present invention provides for the conjugate of the invention, the synthetic virus like particle of the invention, or the pharmaceutical composition of the invention for use as a medicament, preferably for use in a method for preventing an infection or for reducing the risk of an infection, more preferably for use in a method for preventing or reducing the risk of an infection caused by a respiratory syncytial virus. The present invention provides for the conjugate of the invention, the synthetic virus like particle of the invention, or the pharmaceutical composition of the invention for use as a medicament, preferably for use in a method for preventing an infection or for reducing the risk of an infection, more preferably for use in a method for preventing or reducing the risk of an RSV infection.

The invention further relates to a method of eliciting or modulating an immune response or to a method of limiting the risk of developing a disease, preferably an infection, more preferably an infection associated with or caused by an RSV, wherein an immunogenically effective amount of the conjugate or the synthetic virus like particle of the invention is administered to a subject, preferably a human, more preferably a child or elderly people. The invention further relates to a method for treating a disease, preferably an infection, more preferably an infection associated with or caused by an RSV, comprising administering an immunogenically effective amount of the conjugate or the synthetic virus like particle of the invention to a subject, preferably a human, more preferably a child or elderly people. Moreover, the invention relates to the conjugate or the synthetic virus like particle of the invention for use in treating a disease, preferably an infection associated with or caused by an RSV. Moreover, the invention relates to the conjugate or the synthetic virus like particle of the invention for use in treating a disease, preferably an infection caused by an RSV. Moreover, the invention relates to the cyclic peptide, the conjugate or the synthetic virus like particle of the invention for use in treating a disease, preferably an RSV infection.

### EXAMPLES

### Example 1

### Preparation of Lipid Building Blocks

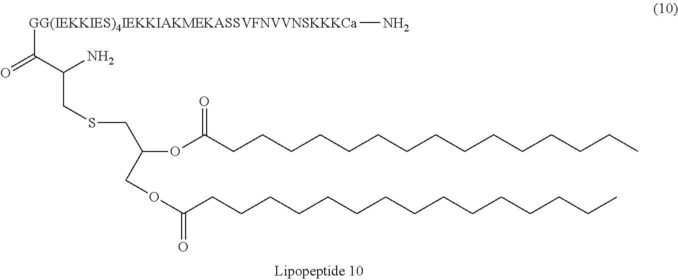

This lipopeptide 10 is based on the IEKKIEA (SEQ ID NO:12). defgabc heptad repeat motif fused to universal T-helper cell epitope SEQ-ID NO:6 as well as extra residues of SEQ ID NO:11) at the C-terminus to improve solubility and stability, and to allow conjugation of an antigen, and via a GG-linker to the S-[2,3-bis(palmitoyloxy)-(2)-propyl]-cysteine. The synthesis of lipopeptide 10 was essentially carried out, and the product was purified by RP-HPLC as described in WO 2008/068017. RP-HPLC (Zorbax C₈ column, 30 to 100% MeCN in H₂O (+0.1% CHOOH) over 10 min): Purity: 95%, t_{R}=4.87 min. MALDI-TOF: m/z calculated for C₃₁₂H₅₅₂N₇₄O₈₅S₃: 6796.4; m/z found: 6797.4 [M+H]⁺.

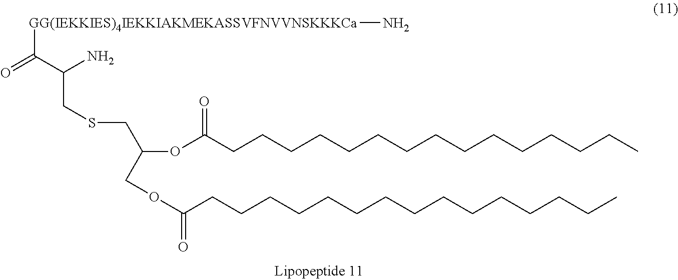

This lipopeptide 11 contains a coiled-coil domain, which has serine in the “c” positions of the heptad repeat “defgabc” IEKKIES (SEQ ID NO:13). The lipopeptide 11 was synthesized and purified by RP-HPLC as described above for lipopeptide 10 and analyzed by analytical RP-HPLC and MALDI-MS. HPLC (Zorbax C₈ column, 30 to 100% MeCN in H₂O (+0.1% CHOOH) over 10 min): Purity: 90%, t_{R}=4.81 min.; MALDI-MS: MW calc. for C₃₁₂H₅₅₂N₇₄O₈₉S₃: 6860.46 Da. found 6860.61 Da (±0.05%).

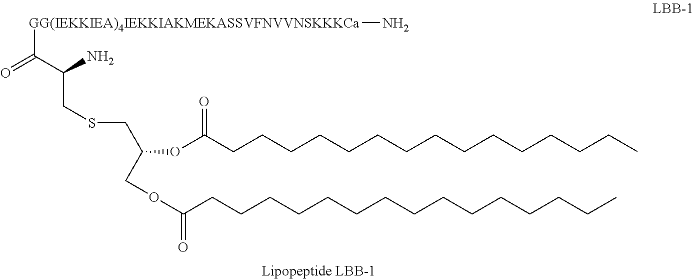

This lipopeptide building block LBB-1 corresponds to lipopeptide 10 except that the lipid Pam₂-Cys has the R-configuration at the chiral 2-propyl carbon atom and the R-configuration of the chiral carbon of the cysteinyl moiety. The building block LBB-1 was analyzed by RP-HPLC RP-HPLC (Agilent VariTide RPC, 0 to 95% MeCN in H₂O (+0.1% TFA) over 63 min.): Purity 97.0%, t_{R}=45.58; ESI-MS MW calculated for C₃₁₂H₅₅₂N₇₄O₈₅S₃: 6796.46 Da. found: 6793.1.

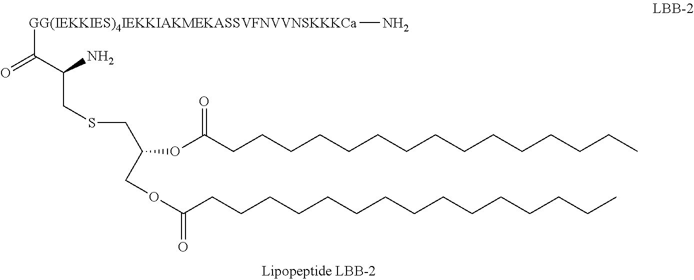

This lipopeptide building block LBB-2 corresponds to lipopeptide 11 except that the lipid Pam₂-Cys has the R-configuration at the chiral 2-propyl carbon atom and the R-configuration of the chiral carbon of the cysteinyl moiety. The building block LBB-2 was analyzed by RP-HPLC (Agilent VariTide RPC, 0 to 95% MeCN in H₂O (+0.1% TFA) over 63 min.): Purity 97.0%, t_{R}=45.58 min. MALDI-MS: MW calc. for C₃₁₂H₅₅₂N₇₄O₈₉S₃: 6860.46 Da. found 6861.1 Da(0.05%).

### Example 2

### Synthesis of Cyclic RSV Peptides and Conjugates Comprising Said Peptides

The present applicants have already described cyclic peptides that are also useful and preferred as antigens for the present inventive conjugates in its application PCT/EP2018/065714 filed on Jun. 13, 2018. In particular, the design and syntheses of such cyclic peptides were described in Example 1 of PCT/EP2018/065714.

Moreover, the preparation of conjugates including such peptides have been described in Example 2 of PCT/EP2018/065714 and its use for immunizations against RSV infections in Examples 3-5 of PCT/EP2018/065714, and hereby in detail, in Example 3 of PCT/EP2018/065714 its use for immunogenicity studies against RSV in mice, in Example 4 of PCT/EP2018/065714 for further intramuscular immunization experiments, and in Example 5 for the generation of monoclonal antibodies.

Not only are the disclosure of PCT/EP2018/065714 incorporated herein in its entirety by way of reference, but all the disclosures of PCT/EP2018/065714, in particular, the disclosure related to the specific cyclic peptides, conjugates and other component moieties, and the generated biological data hereto are specifically incorporated herein in its entirety by way of reference.

It is believed that said biological data and effects described in particular in Examples 3-5 of PCT/EP2018/065714, in view of the disclosed cyclic peptides, conjugates and component moieties, and in particular in view of the described cyclic peptides, conjugates and components of Examples 1-2 of PCT/EP2018/065714, makes it plausible that said biological data and effects are achieved with the lipid building blocks, conjugates and SVLPs of the present invention as well and thus, upon adaption to the present invention and the features of the present invention. It is thus believed that the scope of the invention is further made plausible by reference to said biological data and effects taking the adaption to the present invention and the features of the present invention and the inventive lipid building blocks, conjugates and SVLP hereto into account.

Irrespective of its incorporation by reference of PCT/EP2018/065714, some disclosure thereof is repeated again herein either in full or in summarized form. For the sake of convenience, the numbering of said cyclic peptides, conjugates and other component moieties and their sequences of PCT/EP2018/065714 have been maintained for the present invention and description.

Example 1 of PCT/EP2018/065714 describes maleimido-peptide 1 (SEQ ID NO:14) and (aminooxy)acetyl peptide 2 (SEQ ID NO:15) in particular as reference example since, as disclosed in Example 3 of PCT/EP2018/065714, conjugates comprising said peptides do not generate antibody responses and neutralizing antibodies against RSV A2 and A/Long strains.

The syntheses of said peptides including the cyclic peptides useful and preferred for the present invention were typically carried out using Fmoc solid phase peptide synthesis (SPPS) methods using Tentagel R Ram resin on 0.5 mmol scale. After completion of assembly, the peptides were cleaved from the resin and the side-chain protecting groups were removed, and the peptides purified by RP-HPLC on a preparative column and, typically, lyophilized to afford the peptides as white powders. Subsequently, analysis by RP-HPLC were effected and purity, t_{R}, ESI-MS and MW were determined.

Further preferred cyclic peptides for the present invention, the syntheses and characterization of which were already described in PCT/EP2018/065714 are as follows:

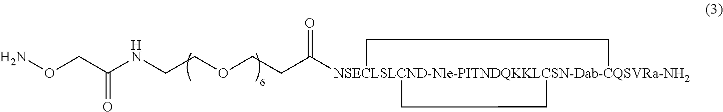

In (aminooxy)acetyl peptide 3 (SEQ ID NO:16), an (aminooxy)acetyl moiety (denoted as “AOAc”) is coupled to the N-terminus of the peptide via a 21-amino-3,6,9,12,15,18-hexaoxaheneicosan-21-oic amide linker, and a D-Ala (denoted as “a”) is coupled to the C-terminus as the amide in order to confer stability to exoproteases. (Aminooxy)acetyl peptide 3 contains two disulfide bonds.

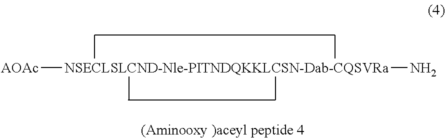

(Aminooxy)acetyl peptide 4 (SEQ ID NO:17) is derived from the F-protein of the RSV virus upon several sequence modifications. An (aminooxy)acetyl moiety (denoted as “AOAc”) is coupled to the N-terminus of the peptide and a D-Ala (denoted as “a”) is coupled to the C-terminus as the amide in order to confer stability to exoproteases. The (aminooxy)acetyl peptide 4 contains two disulfide bonds as depicted in above formula. (Aminooxy)acetyl peptide 4 was assembled by standard Fmoc SPPS. AOAc was coupled as Bis-Boc-aminooxy-acetic acid N-hydroxysuccinimide ester (Boc₂-Aoa-OSu). After completion of assembly the peptide was cleaved from the resin and the side-chain protecting groups were removed by treatment with 87.5% TFA, 5% TA, 5% H₂O, 2.5% EDT for 2.5h. For cyclization by air oxidation, the reduced peptide was dissolved in 0.33 M ammonium bicarbonate buffer, pH 7.8 and stirred overnight. The cyclic peptide was then purified by RP-HPLC on a preparative C18 column and lyophilized to afford 4 as a white powder. Analytical RP-HPLC (Vydac 218TP54, 5 μm, 4.6 mm×250 mm column, 0-60% MeCN in H₂O (+0.1% TFA) over 40 min): Purity: 90.4%; t_{R}=25.07 min. MALDI-MS: MW calculated for C₁₃₄H₂₂₆N₄₄O₄₉S₄: 3365.78 Da; MW found: 3365.80 Da (±0.01%).

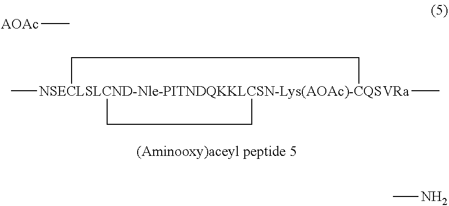

In (aminooxy)acetyl peptide 5 (SEQ ID NO:18), an (aminooxy)acetyl moiety (denoted as “AOAc”) is coupled to a lysine side chain in the peptide, the N-terminus is acetylated and a D-Ala (denoted as “a”) is coupled to the C-terminus. (Aminooxy)acetyl peptide 5 contains two disulfide bonds.

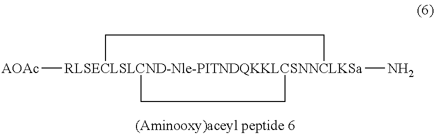

In (aminooxy)acetyl peptide 6 (SEQ ID NO:19), an (aminooxy)acetyl moiety (denoted as “AOAc”) is coupled to the N-terminus of the peptide and a D-Ala (denoted as “a”) is coupled to the C-terminus. (Aminooxy)acetyl peptide 6 contains two disulfide bonds.

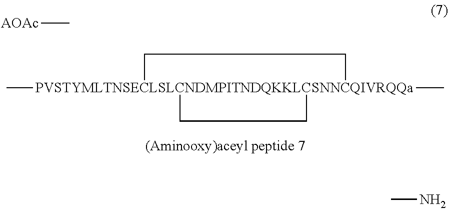

In (aminooxy)acetyl peptide 7 (SEQ ID NO:20), an (aminooxy)acetyl moiety (denoted as “AOAc”) is coupled to the N-terminus and a D-Ala (denoted as “a”) is coupled to the C-terminus. (Aminooxy)acetyl peptide 7 contains two disulfide bonds.

### Preparation of Conjugates for Immunizations

Example 2 of PCT/EP2018/065714 describes the preparation of conjugates comprising the cyclic RSV peptides further by way of a general Scheme A-B-C where “A” denotes the antigen, “B” the linker and “C” the lipopeptide building block, and “X” denotes the attachment to the lipopeptide building block C.

Antigen A1 comprises SEQ ID NO:37, antigen A2 comprises SEQ ID NO:38, antigen A3 comprises SEQ ID NO:2, and antigen A4 comprises SEQ ID NO:3.

### Antigens A:

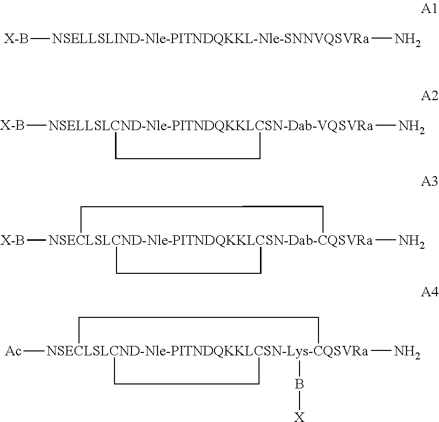

### Linkers B:

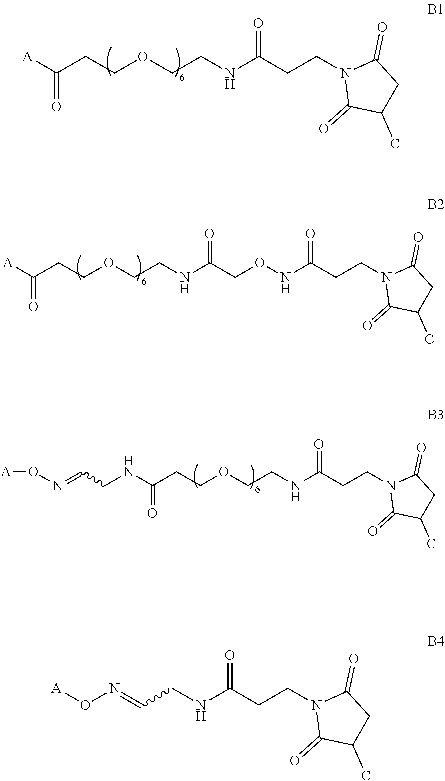

### Lipopeptides Building Blocks C:

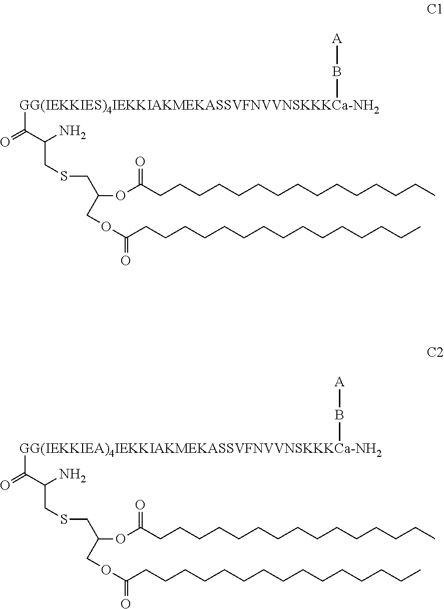

Moreover, further specific linkers, lipopeptides and conjugates are described in Example 2 of PCT/EP2018/065714 which specific disclosure is incorporated herein in its entirety by way of reference and are as follows:

### Maleimide PEG6 Aldehyde Linker Compound 8

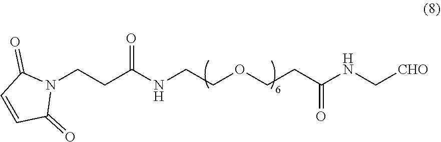

Linker compound 8 was synthesized by reacting SM-PEG₆ (Thermo Fisher Scientific) with aminoacetaldehyde dimethyl acetal in H₂O. SM-PEG6 (7.6 mg, 12.6 μmol) was suspended in 0.3 ml H₂O and 17 μl of a 1:10 (v/v) solution of aminoacetaldehyde dimethyl acetal in H₂O was added. The mixture was stirred for 90 min. at r.t. The cross-linker was purified by RP-HPLC on a C₈ column and lyophilized. ESI-MS: MW calculated for C₂₆H₄₅N₃O₁₂: 591.66; MW found: 591.32 (±0.05%).

For hydrolysis of the dimethyl acetal the linker 8 (20 mg) was treated with 95% TFA, 5% H₂O for 5 min. The TFA was removed in vacuo. ESI-MS C₂₄H₃₉N₃O₁₁: 545.59 Da; MW found: 545.28 Da (±0.05%).

### Maleimide C3 Aldehyde Linker Compound 9

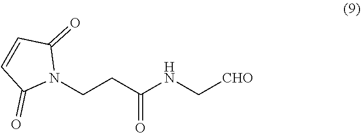 [SMILES: O=CCNC(=O)CCN1C(=O)C=CC1=O]

Lipopeptide 10 and Lipopeptide 11 have already been described in Example 1 above.

### Conjugate 12 (Antigen A3+Linker B3+Lipopeptide Building Block C1)

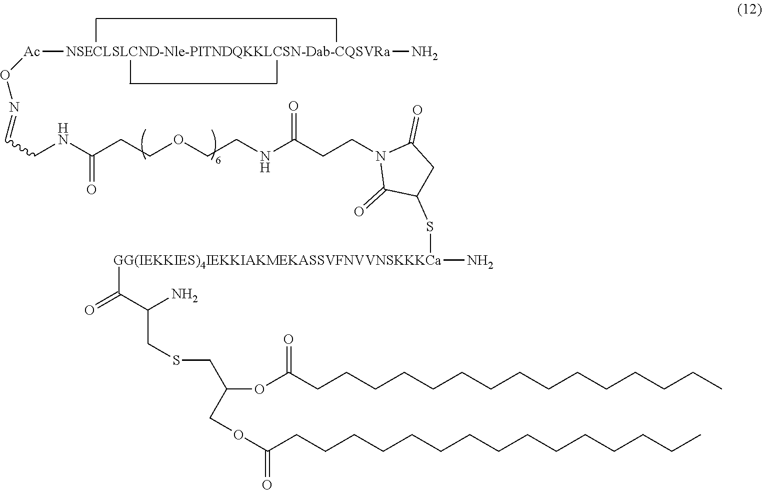

### Conjugate 13 (Antigen A3+Linker B3+Lipopeptide C2)

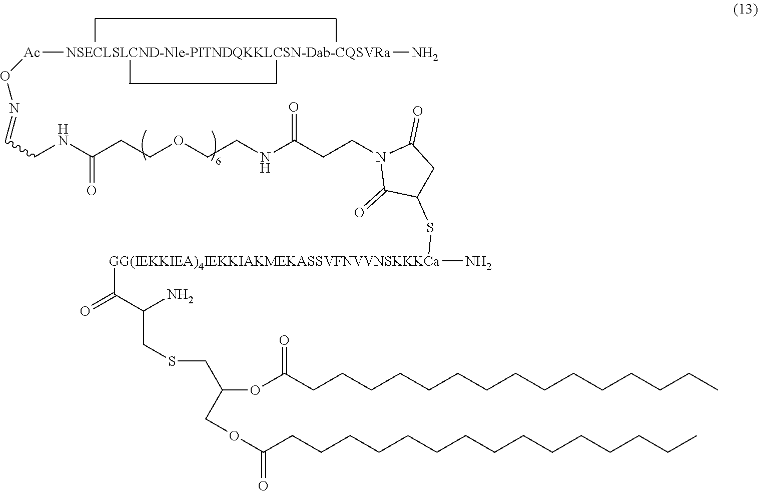

### Conjugate 14 (Antigen A3+Linker B4+Lipopeptide C1)

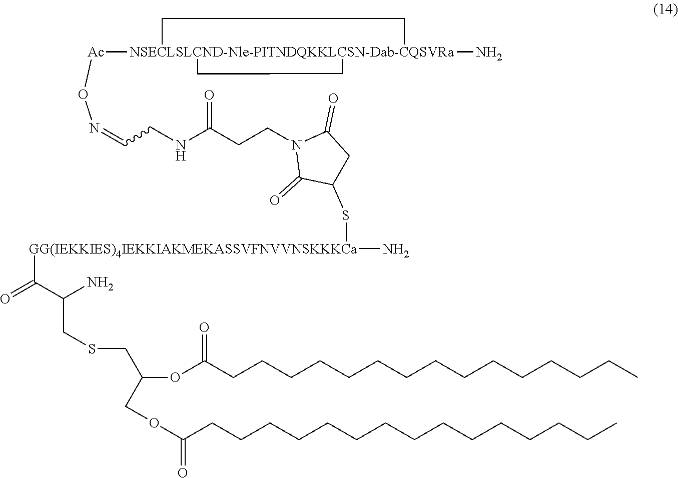

### Conjugate 15 (Antigen A3+Linker B2+Lipopeptide C1)

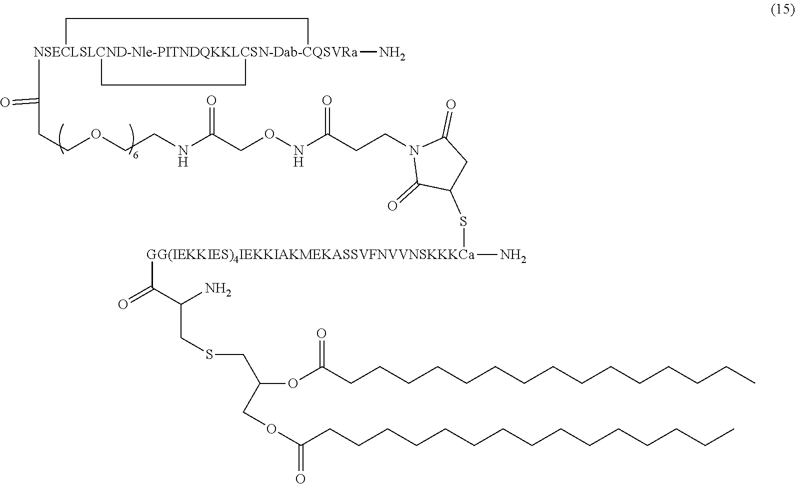

### Conjugate 16 (Antigen A4+Linker B3+Lipopeptide C1)

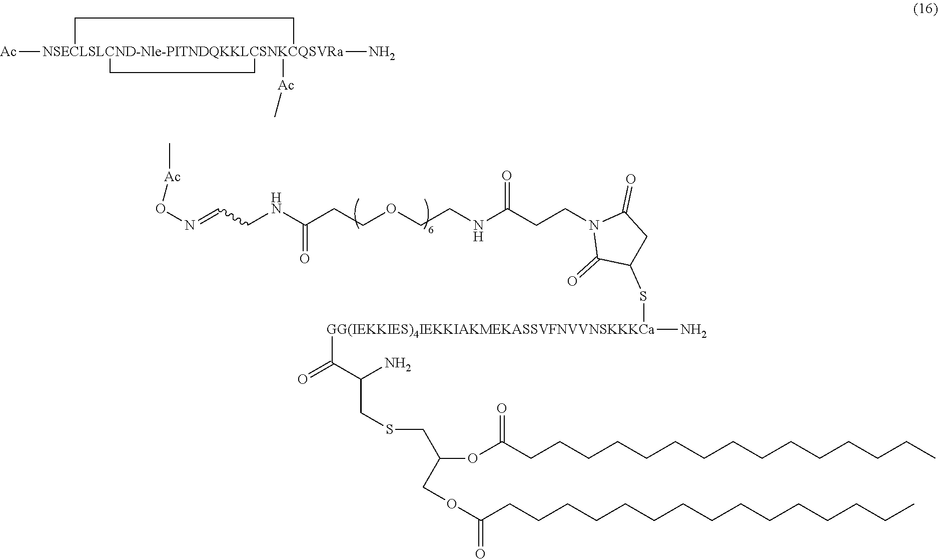

### Conjugate 17 (Antigen A1+Linker B1+Lipopeptide C1).

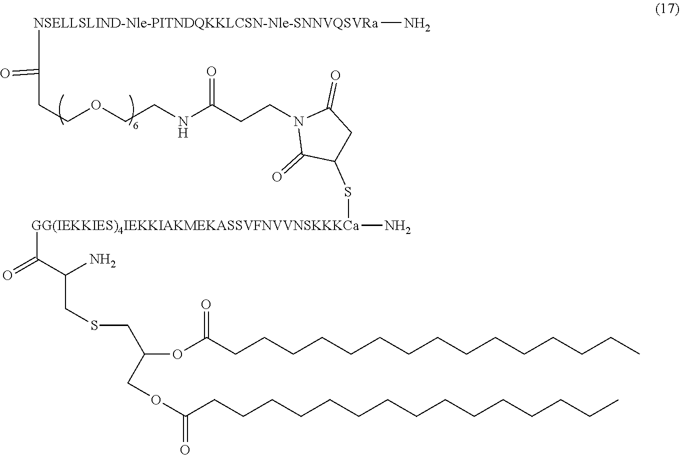

### Conjugate 18 (Antigen A2+Linker B2+Lipopeptide C1)

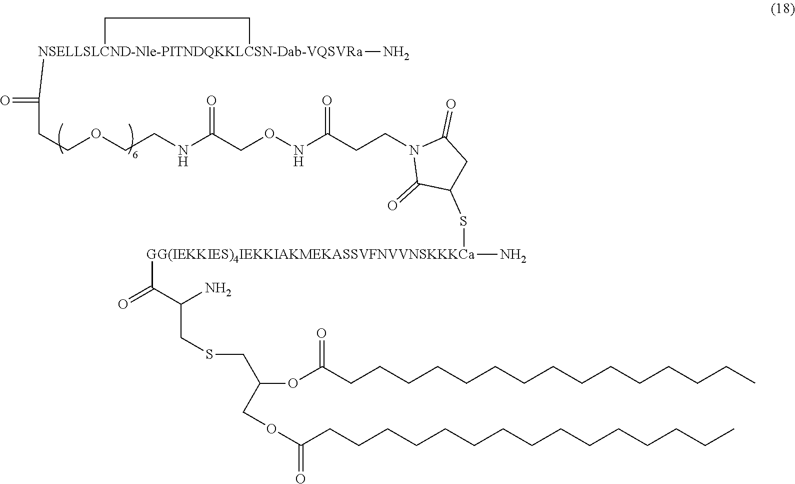

Example 3 of PCT/EP2018/065714 describes immunogenicity studies against RSV in mice, which specific disclosure is incorporated in its entirety herein by way of reference.

The results of Example 3 of PCT/EP2018/065714 demonstrate that immunization with conjugate 12, 13, and 15 elicits high neutralizing titers in serum without co-administration of an adjuvant. Lower but detectable titers were found in sera from animals immunized with conjugate 14 and 16 but not in sera from animals immunized with the corresponding linear or monocyclic peptide antigens (conjugates 17 and 18). Immunization with conjugate 12 leads to full protection of the lungs and reduced pulmonary histopathology after challenge, whereas only a partial reduction of virus in the lungs and strong histopathology is observed upon challenge after immunization with FI-RSV. Immunization with 13 also significantly reduces the virus-replication in the lungs and the pulmonary histopathology.

Example 4 of PCT/EP2018/065714 describes intramuscular immunization experiments, which specific disclosure is incorporated in its entirety herein by way of reference.

The results of Example 4 of PCT/EP2018/065714 shows that animals immunized with 150 μg or 300 μg of conjugate 12 showed a strong reduction (˜3×Log 10) of virus titers compared to the PBS control group. The majority of animals did not have detectable virus in the lungs (Log 10 titer ≤2.6 PFU/g). Animals vaccinated with FT-RSV, 15 μg or 15 μg+Adju-Phos® showed lesser protection (reduction by ˜2×Log 10). The adjuvant did not seem to improve the protection. Animals immunized with PBS depicted the pathology typically associated with a primary RSV infection. Animals vaccinated with FI-RSV showed strong pathology, indicative of vaccine associated disease enhancement. All animals immunized with conjugate 12 showed no signs of vaccine associated disease enhancement and equal or lower pathology scores versus PBS control animals. The results show that two intramuscular immunizations with conjugate 12 without co-administration of an adjuvant are sufficient to high levels of protective neutralizing antibodies and that immunization with conjugate 12 does not cause enhanced respiratory disease over a wide dose range.

Example 5 describes the generation of monoclonal antibodies, which specific disclosure is incorporated in its entirety herein by way of reference.

The results of Example 5 of PCT/EP2018/065714 shows that antibodies with KD's in the sub nano-molar range and neutralizing potency similar or better to that of Palivizumab can be generated in mice after immunization with conjugate 12.

Example 6 describes the generation of sequence variants of the cyclic peptide and their syntheses, which specific disclosure is incorporated in its entirety herein by way of reference.

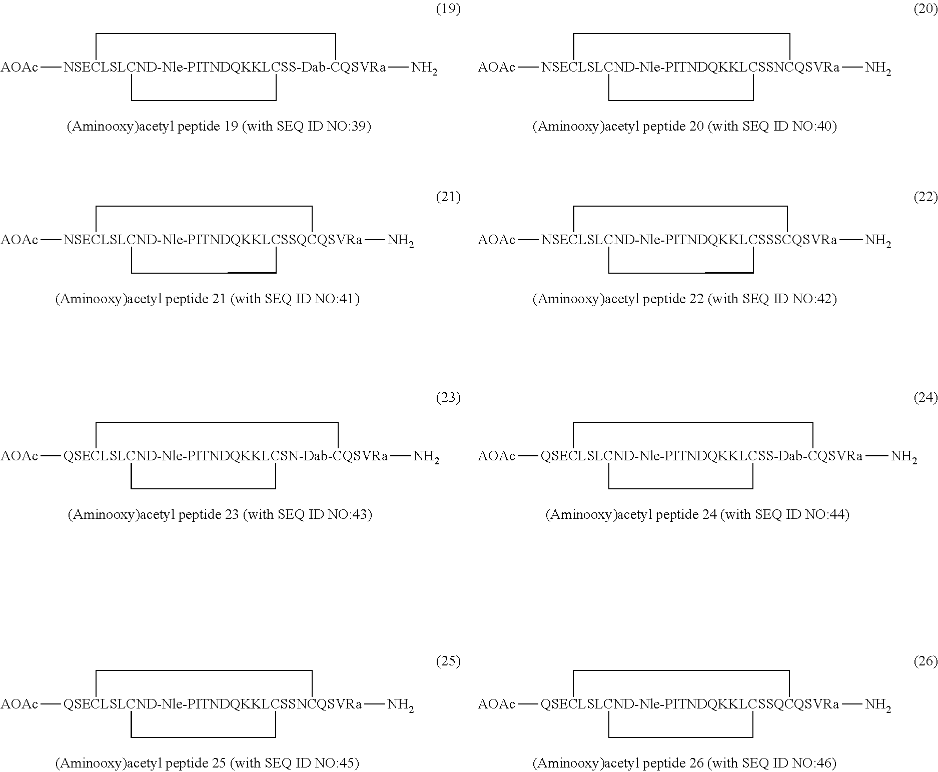

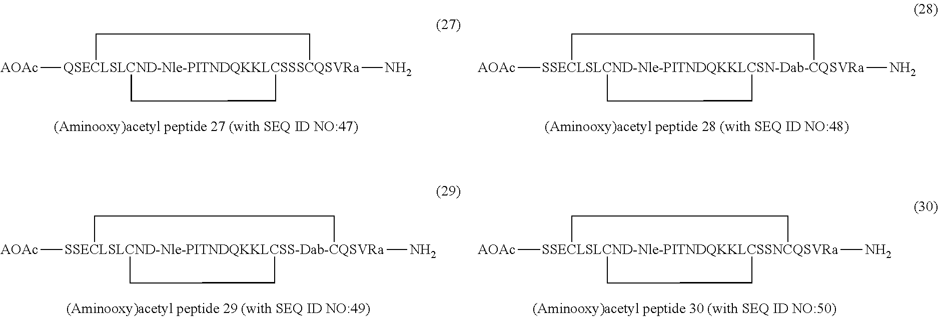

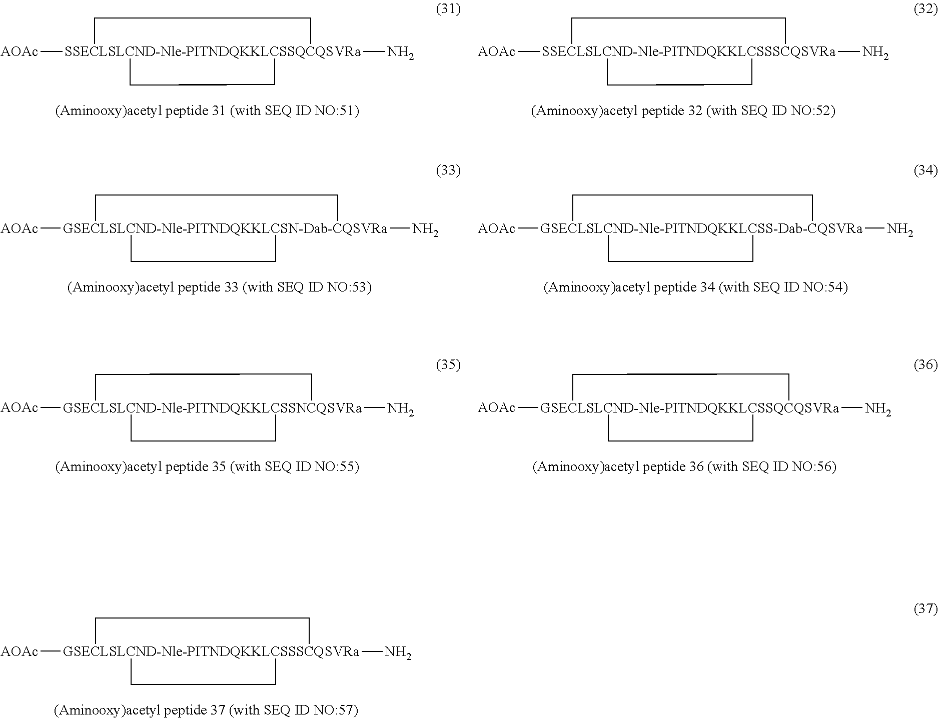

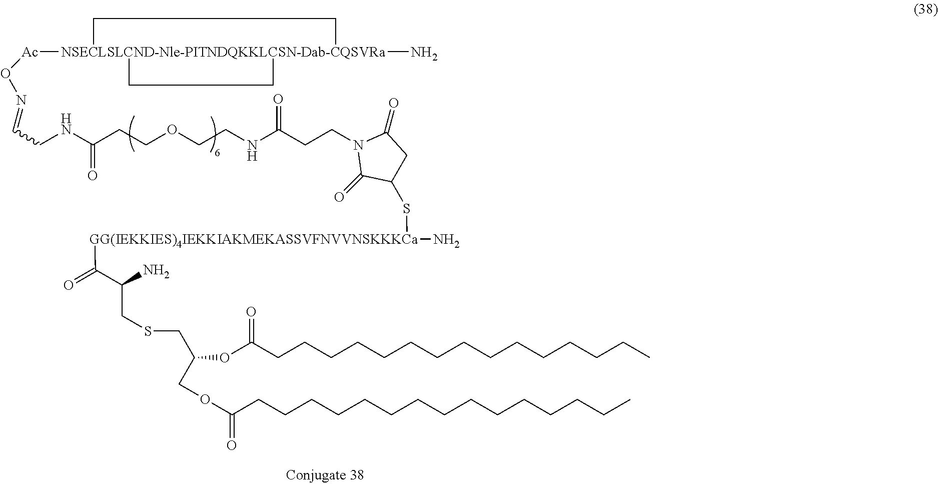

Conjugate 38 was prepared and purified essentially as described below for conjugate 39 except that lipopeptide building block LBB-2 was used instead of LBB-1. Analytical UPLC (ACQUITY UPLC BEH C8, 1.7 μm, 2.1×150 mm 10 to 90% MeCN in H₂O (+0.1% TFA) over 45 min., 40° C.): Purity 79%, t_{R}=32.43 min. MALDI-MS: MW calc. for C₄₇₀H₈₁₅N₁₂₁O₁₄₄S₇: 10689.8 Da. found: 10746.9

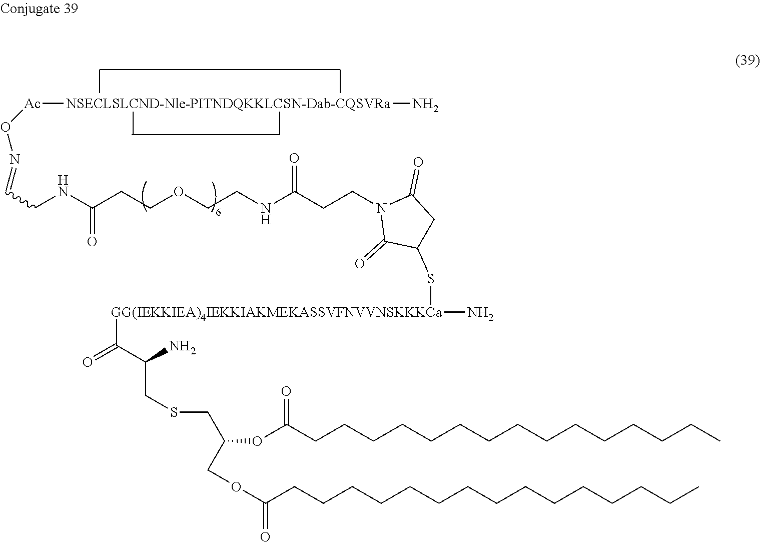

To prepare conjugate 39, a solution of (aminooxy)acetyl peptide 4 (3 mg, 0.9 μmol) in 0.25 ml 0.1 M sodium acetate buffer, pH 3.5 was added to linker 8 (1.9 mg, 3.6 μmol) in 0.25 ml 0.1 M sodium acetate buffer, pH 3.5. The mixture was stirred for 2.5h and the oxime was purified by RP-HPLC on a preparative C8 column. The intermediate was analyzed by analytical UPLC (ACQUITY UPLC BEH C8, 1.7 μm, 2.1×150 mm, 10 to 70% MeCN in H₂O (+0.1% TFA) over 60 min., 70° C.): Purity 95%, t_{R}=16.59 min. ESI-MS: MW calculated for C₁₅₈H₂₆₃N₄₇O₅₉S₄: 3893.35 Da; MW found: 3892.35 (±0.01%). The oxime (2.0 mg, 0.5 μmol) was dissolved in 0.5 ml H₂O and added to a solution of lipopeptide building block LBB-1 (3.1 mg, 0.5 μmol) in 2 ml 50% MeCN. The pH was adjusted to pH=6.5 with 0.1 N NaOH/0.1 N HCl and the mixture was stirred at r.t. for 2.5 h. The conjugate 47 was purified by RP-HPLC on a C8 column. The TFA was removed using AG-X2 anion exchange resin (acetate form). The conjugate was analyzed by analytical UPLC and MS. UPLC (ACQUITY UPLC BEH C8, 1.7 μm, 2.1×150 mm 40 to 80% MeCN in H₂O (+0.1% TFA) 15 over 50 min., 40° C.): Purity 94%, t_{R}=20.75 min. ESI-MS: MW calc. for C₄₇₀H₈₁₅N₁₂₁O₁₄₈S₇: 10753.81 Da. found 10751.1 Da (±0.05%).

### Example 3

### Synthesis of a Malaria Peptide Antigen and Conjugates Comprising Said Peptide

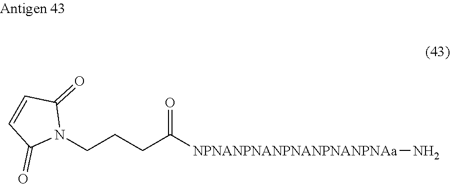

The peptide sequence is taken from the NPNA-repeat region of the circumsporozoite (CS) protein of the malaria parasite Plasmodium falciparum. An extra D-alanine-amide (denoted “a-NH₂”) is added at the C-terminus to improve stability. The gamma-maleimidobutyryl (GMB) group has been added to allow conjugation with a cysteine residue. The synthesis of antigen 43 was essentially carried out, and the product was purified by RP-HPLC as described in WO 2008/068017. Analytical reverse phase RP-HPLC (Zorbax C18 column, 5 to 30% MeCN in H₂O (+0.1% TFA) over 25 min.: purity>95%, t_{R}=13.02 min. LC-MS (Zorbax C18 column, 5 to 100% MeCN in H₂O (+0.1% CHOOH) over 10 min): t_{R}=3.15 min.; ESI-MS m/z=746.5 [M+3H]3+

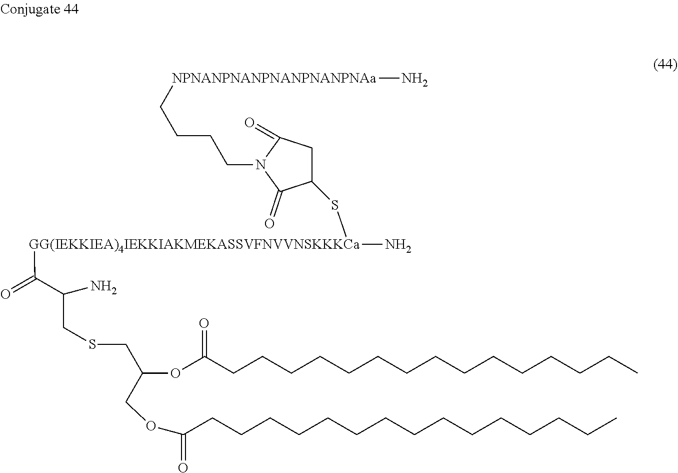

To couple the antigen 43 to the lipopeptide 10, a solution of lipopeptide 10 (6.8 mg, 1.0 μmol) in H₂O/MeCN 1:1 (0.5 ml) was added drop-wise to a stirred solution of 43 (2.7 mg, 1.2 μmol, 1.2 equiv.) in H₂O/MeCN 1:1 (1 ml, 2.7 mg, 1.2 μmol, 1.2 equiv.). The pH was adjusted carefully to pH 6.5 using 0.1 N NaOH and the mixture was stirred for 3 h. After completion of the coupling reaction, the mixture was diluted with H₂O containing 0.10% TFA (2 ml) and the conjugate was purified by reverse phase RP-HPLC on a C4 semi-preparative column (Interchrom) using a gradient of 50 to 100% MeCN in H₂O (+0.1% TFA) over 17 min. Analytical reverse phase RP-HPLC (Interchrom C4 column, 25 to 100% MeCN in H₂O (+0.1% TFA) over 25 min.): purity>97%, t_{R}=19.64 min; MALDI-TOF: m/z calculated for C₄₀₃H₆₈₆N₁₀₆O₁₂₀S₃: 9032.6; m/z found: 9032.2 [M+H]+.

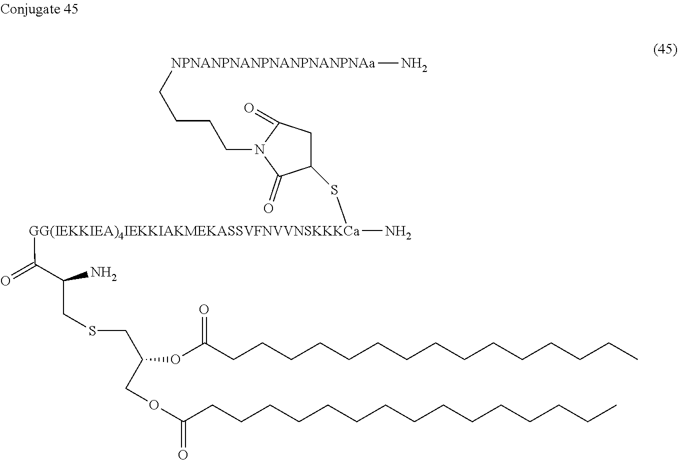

The synthesis and purification of conjugate 45 was essentially performed as described above for conjugate 44, except that LBB-1 was used instead of lipopeptide 10. Analytical reverse phase RP-HPLC (Interchrom C4 column, 25 to 100% MeCN in H₂O (+0.1% TFA) over 25 min.): purity>97%, t_{R}=19.64 min. MALDI-TOF: m/z calculated for C₄₀₃H₆₈₆N₁₀₆O₁₂₀S₃: 9032.6; m/z found: 9032.4 [M+H]+

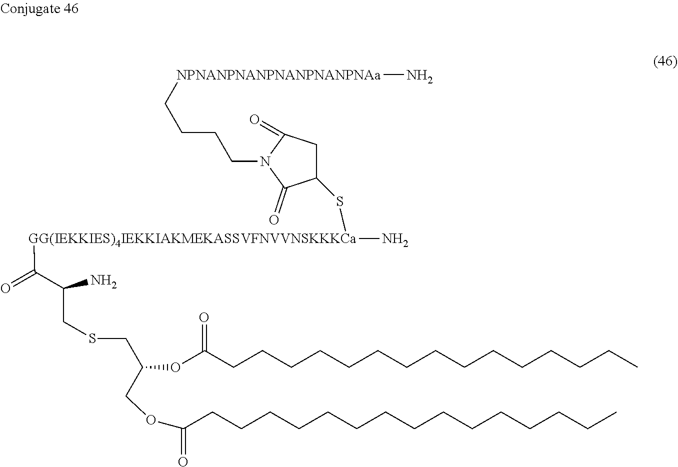

The synthesis and purification of conjugate 46 was essentially performed as described above for conjugate 44, except that LBB-2 was used instead of lipopeptide 10. UPLC (ACQUITY UPLC BEH C8, 1.7 μm, 2.1×150 mm, 10 to 90% MeCN in H₂O (+0.1% TFA) over 10 min., 25° C.): Purity >79%, t_{R}=35.76 min.): purity 90%, t_{R}=8.62 min.; MALDI-TOF: m/z calculated for C₄₀₃H₆₈₇N₁₀₇O₁₂₃S₃: 9095.74; m/z found: 9096.6

### Example 4

### Avidity Index of Conjugates Comprising a Malaria Peptide Antigen

For mouse immunogenicity studies, Balb/c mice (5 per group) were s.c. immunized with the formulations depicted in Table 2.

TABLE 2
Formulations

| No. | Description | Dose | Adjuvant | Route |
|---|---|---|---|---|
| 1 | 44 in 0.1 ml PBS | 40 μg | none | SC |
| 2 | 45 in 0.1 ml PBS | 40 μg | none | SC |
| 3 | 46 in 0.1 ml PBS | 40 μg | none | SC |

Immunizations where performed on days 0, 28 and 56. Blood was collected on day 66. Relative avidity indices were determined by KSCN elution ELISA essentially as described (Perciani et al, J Clin Lab Anal. 2007; 21(3):201-6) using 2 M KSCN for elution.

IgG endpoint dilution ELISA were essentially performed as in WO 2008/068017. For the KSCN elution ELISA, Nunc Immunoplates Polysorb F96) where coated over night with 50 μl of a 5 μg/ml antigen solution in PBS, washed 3× with 150 μl PBS containing 0.05% TWEEN® 20 and incubated for 2.5h with duplicates of two-fold serum dilutions in PBS containing 0.5% skim milk powder and 0.05% TWEEN® (50 μl per well) at room temperature. Plates were washed three times with PBS containing 0.05% TWEEN® 20 as described above. One half of the wells were incubated with 2 M KSCN in PBS, pH 7.2 for 15 min and the other half of the wells was incubated with plain PBS for the same amount of time. The plates were then washed, incubated with 50 μl of alkaline phosphatase conjugated rabbit anti-mouse IgG antibody (Sigma, gamma chain specific), diluted 1:20′000 in PBS containing 0.5% skim milk powder and 0.05% TWEEN® 20 for 1 h at room temperature, washed again three times with PBS+0.05% TWEEN® 20 and incubated in the dark with 50 μl of a 1 mg/ml solution of p-nitrophenyl phosphate (Sigma) in 50 mM sodium carbonate, 1 mM MgCl2, pH 9.6 at room temperature. The absorbance was read after appropriate time at 405 nm on a SpectraMax M5 microplate reader. The avidity index was then calculated for each serum as ratio between the area under the curves obtained for the KSCN treated wells and the area under the curves obtained for the corresponding controls (Perciani et al, J Clin Lab Anal. 2007; 21(3):201-6).

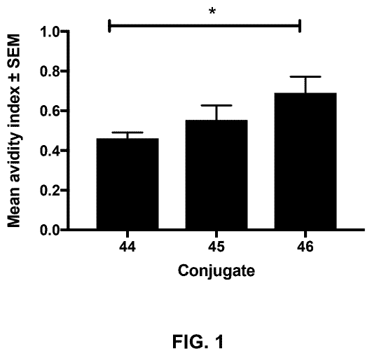

Mean log 10 IgG ELISA endpoint dilution titers and mean avidity indices ±one standard error of the mean are shown in Table 3 and FIG. 1.

TABLE 3
Mean Avidity index.

| | | Log 10 Mean ELISA | Mean Avidity |
|---|---|---|---|
| No. | Description | endpoint titer ± SEM | Index ± SEM |
| 1 | 44 in 0.1 ml PBS | 5.05 ± 0.08 | 0.46 ± 0.03 |
| 2 | 45 in 0.1 ml PBS | 5.06 ± 0.14 | 0.55 ± 0.07 |
| 3 | 46 in 0.1 ml PBS | 4.18 ± 0.12 | 0.69 ± 0.08 |

The results show that immunization with conjugate 45 comprising the heptad repeat motif IEKKIEA and the lipid Pam₂Cys with the (R)-configuration at the 2-propyl carbon without co-administration of an adjuvant leads to higher avidity antibodies than immunization with 44 which comprises a mixture of (R)- and (S)-diastereomers at the the 2-propyl carbon, and further show that immunization with conjugate 46, which comprises IEKKIES instead of IEKKIEA leads to an even higher mean avidity index (P=0.0291) despite a significantly lower endpoint dilution titer (P=0.0011) vs. 44.

### Example 5

### Avidity Index of Conjugates Comprising a RSV Antigen

For mouse immunogenicity studies, Balb/c mice (5 per group) were immunized with the formulations in Table 4.

TABLE 4
Formulations

| No. | Description | Dose | Adjuvant | Route |
|---|---|---|---|---|
| 1 | 47 in 0.1 ml PBS | 50 μg | none | SC |
| 2 | 38 in 0.1 ml PBS | 50 μg | none | SC |

Immunizations where performed on days 0, 28 and 56. Blood was collected on day 66. Relative avidity indices were determined by KSCN elution ELISA essentially as described (Perciani et al, J Clin Lab Anal. 2007; 21(3):201-6) using 2 M KSCN for elution.

Mean avidity indices±one standard error of the mean are shown in Table 5.

TABLE 5
Mean Avidity index.

| | | Mean Avidity |
|---|---|---|
| No. | Description | Index ± SEM |
| 1 | 47 in 0.1 ml PBS | 0.58 ± 0.06 |
| 2 | 38 in 0.1 ml PBS | 0.64 ± 0.02 |

### Example 6

### Immunogenicity and Efficacy of Two Subcutaneous Administrations of Conjugate 38 in Mice

For this experiment 6-8 weeks old female BALB/c mice (ten animals per group) were immunized two times by the subcutaneous route with 150 μg of conjugate 38 in 0.1 ml PBS (Table 6, No. 1). Two control groups (ten animals per group) were immunized two times with FI-RSV and PBS, respectively (Table 6, No. 2 and 3).

TABLE 6
Formulations

| No. | Description | Dose | Adjuvant | Route |
|---|---|---|---|---|
| 1 | 38 in 0.1 ml PBS | 150 μg | none | SC |
| 2 | FI-RSV | 1:100 | none | SC |
| 3 | PBS | 0 | none | SC |

Animals were immunized on days 0 and 21. On day 42 (D42) all animals where bled and further challenged intranasally with 10⁶ pfu RSV A2 live virus. Five days later, on day 47 (D47), animals were sacrificed, totally bled and the lungs were harvested and bi-sected for viral titrations and histopathology analysis.

Sera from DO and D42 (of animals bled previous to challenge) of the groups immunized with conjugate 38, FI-RSV and PBS were analyzed by Plaque Reduction Neutralization Test (PRNT) for neutralizing antibodies against RSV A2 strain. Test sera was heat inactivated 30 min. at 56° C., diluted 1:10 with EMEM and serially diluted further 1:4. Diluted serum samples were incubated with equal volumes of RSV A2 (25-50 PFU) for 1 hour at room temperature and inoculated in duplicates onto confluent HEp-2 monolayers in 24 well plates. After one hour of incubation at 37° C. in a 5% CO₂ incubator, the wells were overlaid with 0.75% Methylcellulose medium. After 4 days of incubation, the overlays were removed, and the cells were fixed and stained with 0.1% crystal violet for one hour and then rinsed and air-dried. Neutralization titers were calculated as reciprocal serum dilutions required to reach 60% plaque reduction versus virus control wells. The Log 2 virus PRNT titer for RSV A2 is summarized in Table 7.

TABLE 7
Induction of Neutralizing antibodies.

| | | PRNT titer (log₂) | PRNT titer (log₂) |
|---|---|---|---|
| No. | Description | D0 | D42 |
| 1 | 150 μg 38 in PBS | <4.32 | 7.32 ± 0.54 |
| 2 | FI-RSV | <4.32 | <4.32 |
| 3 | PBS | <4.32 | <4.32 |

Two immunizations with conjugate 38 induced high titers of neutralizing antibodies at a dose of 150 μg. The FI-RSV and PBS lead to titers of neutralizing antibodies below the limit of detection (<4.32)

For viral titrations, lung homogenates from D47 were clarified by centrifugation and diluted in EMEM. Confluent HEp-2 monolayers were infected in duplicates with diluted homogenates in 24 well plates. After one hour incubation at 37° C. in a 5% CO2 incubator, the wells were overlaid with 0.75% methylcellulose medium. After 4 days of incubation, the overlays were removed, and the cells were fixed and stained with 0.1% crystal violet for one hour and then rinsed and air dried. Plaques were counted and plaque forming units per gram of tissue were calculated. The geometric mean virus titers are summarized below in Table 8 for D47 (5 days after challenge) samples.

For pulmonary histopathology analysis, lungs were dissected, formalin fixed and embedded in paraffin as described above. Lung sections were stained with hematoxylin and eosin (H&E) for analysis of the four parameters of pulmonary inflammation: peribronchiolitis, perivasculitis, interstitial pneumonia, and alveolitis. Slides were scored blind on a 0-4 severity scale. The scores were subsequently converted to a 0-100% histopathology scale. Average pathology scores are summarized below in Table 8 for D47 samples.

TABLE 8
Lung virus titers and pathology scores on Day 47.

| | | Lung virus titer | Avg. pathology score |
|---|---|---|---|
| | | D47 | D47 |
| No. | Description | (Log10 PFU/g) | (0-100%) |
| 2 | 150 μg 38 in PBS | 2.58 ± 0.81 | 13.33 ± 3.33 |
| 5 | FI-RSV | 3.95 ± 1.24 | 91.25 ± 3.77 |
| 6 | PBS | 4.76 ± 1.50 | 26.25 ± 6.41 |

All animals immunized with PBS showed maximal viral titers in the lung five days post challenge (mean titer ˜4.76 Log 10 PFU/g). Animals immunized with 150 μg of conjugate 38 showed a strong reduction (mean titer ˜2.58 Log 10 PFU/g) of virus titers and the majority of animals did not have detectable virus in the lungs (Log 10 titer; 2.6 PFU/g). Animals vaccinated with FI-RSV showed lesser protection (mean titer ˜3.95 Log 10 PFU/g). On the histopathological level, Animals immunized with PBS depicted the pathology typically associated with a primary RSV infection. Animals vaccinated with FI-RSV showed strong pathology, indicative of vaccine associated disease enhancement. All animals immunized with conjugate 38 showed no signs of vaccine associated disease enhancement and equal or lower pathology scores versus PBS control animals (Table 8).

The results show that two subcutaneous immunizations with conjugate 38 without co-administration of an adjuvant are sufficient to elicit high titers of protective neutralizing antibodies, to protect against RSV A2 virus replication in the lung and the immunization with conjugate 38 does not cause enhanced respiratory disease.

1. A lipopeptide building block consisting of:
(i) a peptide moiety comprising a coiled coil peptide chain segment, wherein said coiled coil peptide chain segment comprises 3 to 8 repeat units, and wherein said repeat unit consists of the sequence IEKKIE-X0 (SEQ ID NO:58), wherein X0 represents an amino acid;
(ii) a lipid moiety comprising the formula LM-I 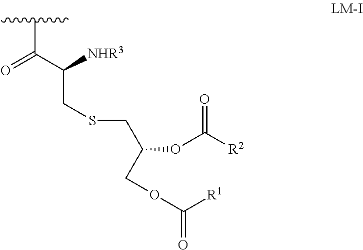
wherein R¹ and R² are independently C₁₁₋₁₅alkyl and wherein R³ is hydrogen or —C(O)C₁₁₋₁₅alkyl;
and wherein said lipid moiety is linked to said peptide moiety, wherein the wavy line in formula LM-I indicates the linkage site to said peptide moiety.

2. The lipopeptide building block of claim 1, wherein said coiled coil peptide chain segment of said peptide moiety consists of 4 repeat units.

3. The lipopeptide building block of claim 1, wherein said coiled coil peptide chain segment consists of the sequence (IEKKIES)₄ (SEQ ID NO:62).

4. The lipopeptide building block of claim 1, wherein said lipid moiety consists of the formula LM-I* 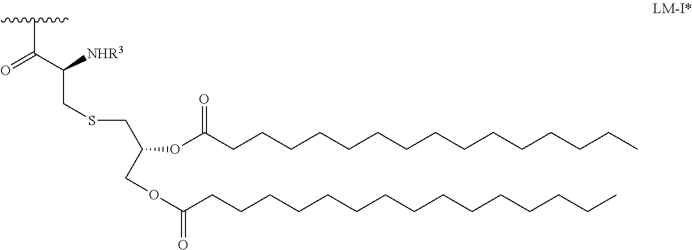
wherein R³ is hydrogen or —C(O)C₁₁₋₁₅alkyl.

5. The lipopeptide building block of claim 1, wherein said lipid moiety consists of the formula LM-I*1:

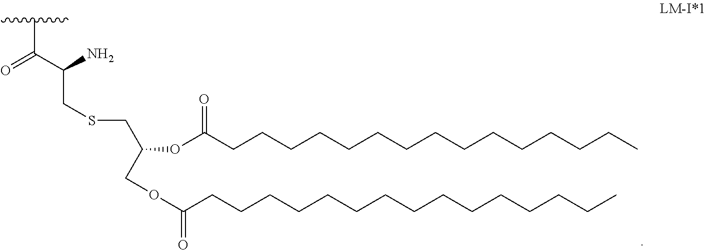 [SMILES: *C(=O)[C@@H](N)CSC[C@@H](COC(=O)CCCCCCCCCCCCCCC)OC(=O)CCCCCCCCCCCCCCC]

6. The lipopeptide building block of claim 1, wherein said lipid moiety and said peptide moiety are linked directly or via a coupling moiety, wherein said coupling moiety is an amino acid linker.

7. The lipopeptide building block of claim 1, wherein said peptide moiety further comprises a T-helper cell epitope.

8. The lipopeptide building block of claim 1, wherein said lipopeptide building block is of the formula LBB-2:

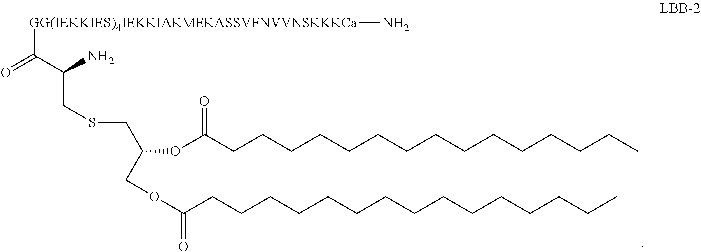

9. A conjugate comprising
(a) a lipopeptide building block of claim 1, and
(b) an antigen,
wherein said antigen is connected, directly or via a linker, to said lipopeptide building block.

10. The conjugate of claim 9, wherein said antigen is a tumor antigen, a self-antigen, a polypeptide of a pathogen, an allergen, or a hapten.

11. The conjugate of claim 9, wherein said conjugate is selected from any one of the formulae (38), (39), (40), (41) or (42):

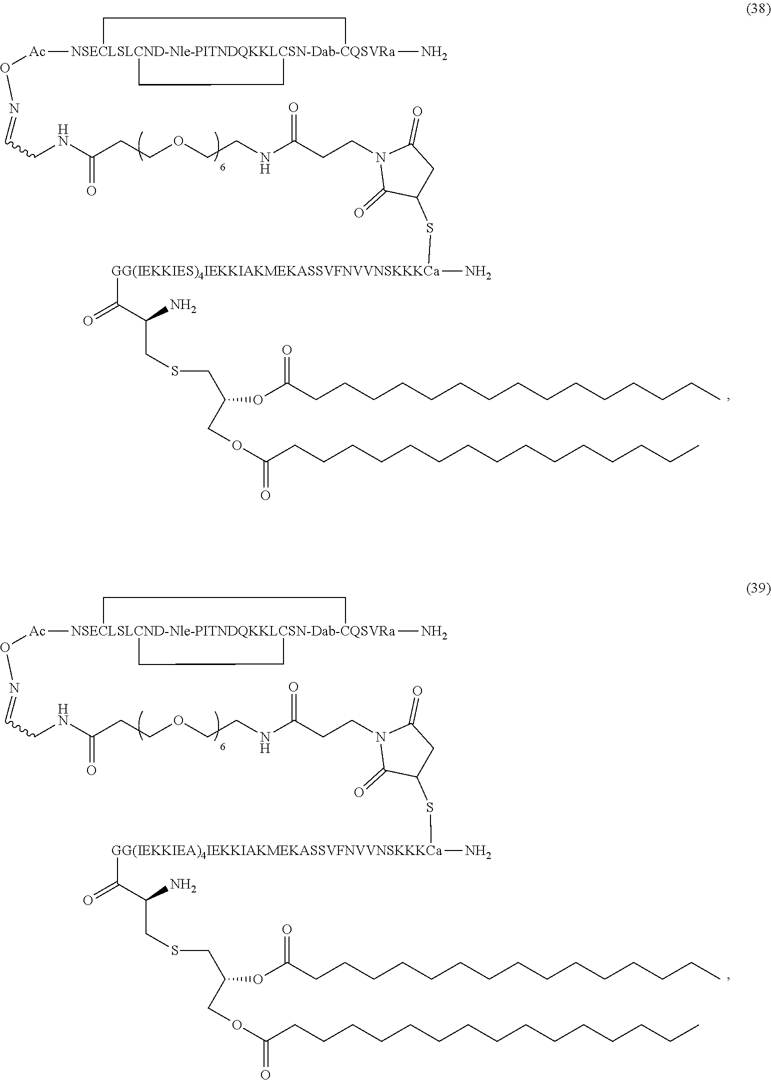

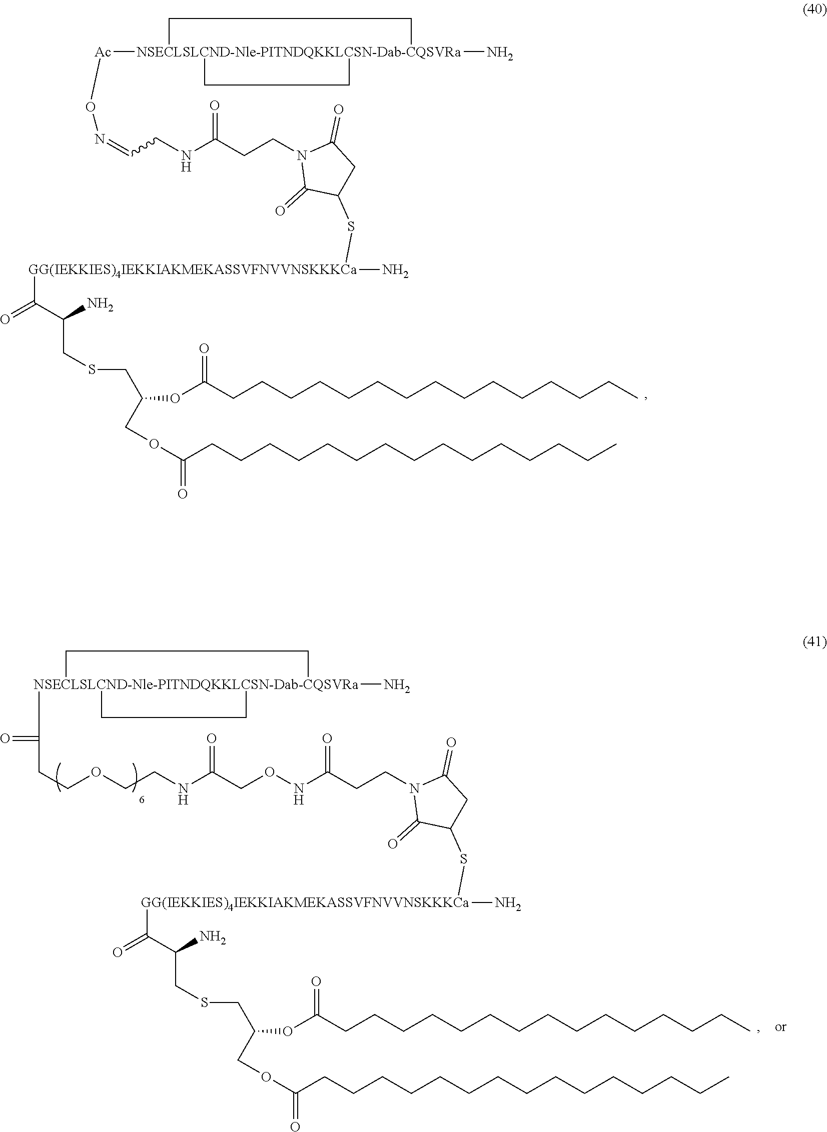

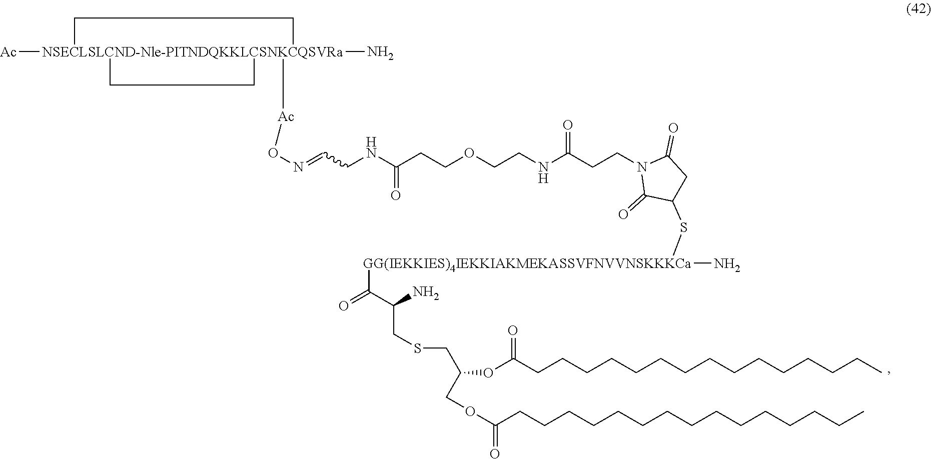

12. A bundle of conjugates comprising 2, 3, 4, 5, 6 or 7 conjugates of claim 9.

13. A synthetic virus-like particle comprising at least one bundle of conjugates of claim 12.

14. A pharmaceutical composition comprising an immunologically effective amount of the conjugate of claim 9, together with a pharmaceutically acceptable diluent, carrier or excipient.

15. A method of reducing the risk of a disease or treating a disease in a subject in need thereof, the method comprising administering to the subject an immunogenically effective amount of a conjugate of claim 9.

16. A pharmaceutical composition comprising an immunologically effective amount of the synthetic virus like particle of claim 13, together with a pharmaceutically acceptable diluent, carrier or excipient.

17. The lipopeptide building block of claim 1, wherein said repeat unit consists of a sequence selected from IEKKIEG (SEQ ID NO:59), IEKKIEA (SEQ ID NO:12), and IEKKIES (SEQ ID NO:13).

18. The lipopeptide building block of claim 1, wherein said repeat unit consists of the sequence IEKKIES (SEQ ID NO:13).

19. The lipopeptide building block of claim 1, wherein said lipid moiety is linked to the N-terminus of said peptide moiety.

20. The lipopeptide building block of claim 2, wherein said 4 repeat units are consecutively linked to each other.


SEQUENCE LISTING (88 sequences)

SEQ ID NO: 1 - protein, 30 aa, synthetic construct
  REGION 1..30: note = Description of Artificial Sequence: Synthetic polypeptide
  VARIANT 1..3: note = Any amino acid
  VARIANT 5..7: note = Any amino acid
  VARIANT 9..11: note = Any amino acid
  VARIANT 22..24: note = Any amino acid
  VARIANT 26..29: note = Any amino acid
  VARIANT 30: note = Any amino acid or absent
XXXCXXXCXXXPITNDQKKLCXXXCXXXXX

SEQ ID NO: 2 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
NSECLSLCNDXPITNDQKKLCSNXCQSVRX

SEQ ID NO: 3 - protein, 30 aa, synthetic construct
  REGION 1..30: note = Description of Artificial Sequence: Synthetic polypeptide
  MOD_RES 11: note = Nle
  MOD_RES 30: note = D-alanine
NSECLSLCNDXPITNDQKKLCSNKCQSVRX

SEQ ID NO: 4 - protein, 30 aa, synthetic construct
  REGION 1..30: note = Description of Artificial Sequence: Synthetic polypeptide
  MOD_RES 12: note = Nle
  MOD_RES 30: note = D-alanine
RLSECLSLCNDXPITNDQKKLCSNNCLKSX

SEQ ID NO: 5 - protein, 40 aa, synthetic construct
  REGION 1..40: note = Description of Artificial Sequence: Synthetic polypeptide
  MOD_RES 40: note = D-alanine
PVSTYMLTNSECLSLCNDMPITNDQKKLCSNNCQIVRQQX

SEQ ID NO: 6 - protein, 20 aa, synthetic construct
  REGION 1..20: note = Description of Artificial Sequence: Synthetic peptide
  REGION 1..20: note = T helper cell epitope
IEKKIAKMEKASSVFNVVNS

SEQ ID NO: 7 - protein, 10 aa, synthetic construct
  REGION 1..10: note = Description of Artificial Sequence: Synthetic peptide
  REGION 1..10: note = FSL-1
  BINDING 1: note = S-(2,3-bispalmitoyloxypropyl)
CGDPKHPKSF

SEQ ID NO: 8 - protein, 10 aa, synthetic construct
  REGION 1..10: note = Description of Artificial Sequence: Synthetic peptide
  REGION 1..10: note = FSL-2
  BINDING 1: note = S-(2,3-bispalmitoyloxypropyl)
CGDPKHPKSR

SEQ ID NO: 9 - protein, 10 aa, synthetic construct
  REGION 1..10: note = Description of Artificial Sequence: Synthetic peptide
  REGION 1..10: note = FSL-3
  BINDING 1: note = S-(2,3-bisstearyloxypropyl)
CGDPKHPKSF

SEQ ID NO: 10 - protein, 14 aa, synthetic construct
  REGION 1..14: note = Description of Artificial Sequence: Synthetic peptide
  REGION 1..14: note = MALP-2
  BINDING 1: note = S-(2,3-bispalmitoyloxypropyl)
CGNNDESNISFKEK

SEQ ID NO: 11 - protein, 5 aa, synthetic construct
  REGION 1..5: note = Description of Artificial Sequence: Synthetic peptide
  REGION 1..5: note = C-terminal sequence
  MOD_RES 5: note = D-alanine
KKKCX

SEQ ID NO: 12 - protein, 7 aa, synthetic construct
  REGION 1..7: note = Description of Artificial Sequence: Synthetic peptide
  REGION 1..7: note = heptad motif
IEKKIEA

SEQ ID NO: 13 - protein, 7 aa, synthetic construct
  REGION 1..7: note = Description of Artificial Sequence: Synthetic peptide
  REGION 1..7: note = heptad motif
IEKKIES

SEQ ID NO: 14 - protein, 30 aa, synthetic construct
  REGION 1..30: note = Description of Artificial Sequence: Synthetic polypeptide
  BINDING 1: note = Maleimido moiety
  MOD_RES 11: note = Nle
  MOD_RES 21: note = Nle
  MOD_RES 30: note = D-alanine
  MOD_RES 30: note = AMIDATION
NSELLSLINDXPITNDQKKLXSNNVQSVRX

SEQ ID NO: 15 - protein, 30 aa, synthetic construct
  REGION 1..30: note = Description of Artificial Sequence: Synthetic polypeptide
  BINDING 1: note = AoAc miety incl. a 1-amino-3,6,9,12,15,18-hexaoxaheneicosan-21- oic amide linker
  MOD_RES 11: note = Nle
  MOD_RES 24: note = Dbu
  MOD_RES 30: note = D-alanine
  MOD_RES 30: note = AMIDATION
NSELLSLCNDXPITNDQKKLCSNXVQSVRX

SEQ ID NO: 16 - protein, 30 aa, synthetic construct; identical to SEQ ID NO: 2
  REGION 1..30: note = Description of Artificial Sequence: Synthetic polypeptide
  BINDING 1: note = AoAc moiety incl. 1-amino-3,6,9,12,15,18-hexaoxaheneicosan-21-oic amide linker
  MOD_RES 11: note = Nle
  MOD_RES 24: note = Dbu
  MOD_RES 30: note = D-alanine
  MOD_RES 30: note = AMIDATION

SEQ ID NO: 17 - protein, 30 aa, synthetic construct; identical to SEQ ID NO: 2
  REGION 1..30: note = Description of Artificial Sequence: Synthetic polypeptide
  BINDING 1: note = AoAc moiety
  MOD_RES 11: note = Nle
  MOD_RES 24: note = Dbu
  MOD_RES 30: note = D-alanine
  MOD_RES 30: note = AMIDATION

SEQ ID NO: 18 - protein, 30 aa, synthetic construct; identical to SEQ ID NO: 3
  REGION 1..30: note = Description of Artificial Sequence: Synthetic polypeptide
  BINDING 1: note = Ac moiety
  MOD_RES 11: note = Nle
  BINDING 24: note = AOAc moiety
  MOD_RES 30: note = D-alanine
  MOD_RES 30: note = AMIDATION

SEQ ID NO: 19 - protein, 30 aa, synthetic construct; identical to SEQ ID NO: 4
  REGION 1..30: note = Description of Artificial Sequence: Synthetic polypeptide
  BINDING 1: note = AOAc
  MOD_RES 12: note = Nle
  MOD_RES 30: note = D-alanine
  MOD_RES 30: note = AMIDATION

SEQ ID NO: 20 - protein, 40 aa, synthetic construct; identical to SEQ ID NO: 5
  REGION 1..40: note = Description of Artificial Sequence: Synthetic polypeptide
  BINDING 1: note = AOAc
  MOD_RES 40: note = D-alanine
  MOD_RES 40: note = AMIDATION

SEQ ID NO: 21 - protein, 30 aa, synthetic construct
  REGION 1..30: note = Description of Artificial Sequence: Synthetic polypeptide
  MOD_RES 11: note = Nle
  MOD_RES 30: note = D-alanine
NSECLSLCNDXPITNDQKKLCSNNCQSVRX

SEQ ID NO: 22 - protein, 30 aa, synthetic construct
  REGION 1..30: note = Description of Artificial Sequence: Synthetic polypeptide
  MOD_RES 11: note = Nle
  MOD_RES 30: note = D-alanine
NSECLSLCNDXPITNDQKKLCSNDCQSVRX

SEQ ID NO: 23 - protein, 30 aa, synthetic construct; identical to SEQ ID NO: 2
  REGION 1..30: note = Description of Artificial Sequence: Synthetic polypeptide
  MOD_RES 11: note = Nle
  MOD_RES 24: note = Orn
  MOD_RES 30: note = D-alanine

SEQ ID NO: 24 - protein, 30 aa, synthetic construct
  REGION 1..30: note = Description of Artificial Sequence: Synthetic polypeptide
  MOD_RES 12: note = Nle
  MOD_RES 30: note = D-alanine
RLSECLSLCNDXPITNDQKKLCSNKCLKSX

SEQ ID NO: 25 - protein, 30 aa, synthetic construct
  REGION 1..30: note = Description of Artificial Sequence: Synthetic polypeptide
  MOD_RES 12: note = Nle
  MOD_RES 25: note = Dbu
  MOD_RES 30: note = D-alanine
RLSECLSLCNDXPITNDQKKLCSNXCLKSX

SEQ ID NO: 26 - protein, 40 aa, synthetic construct
  REGION 1..40: note = Description of Artificial Sequence: Synthetic polypeptide
  MOD_RES 40: note = D-alanine
PVSTYMLTNSECLSLCNDMPITNDQKKLCSNKCQIVRQQX

SEQ ID NO: 27 - protein, 40 aa, synthetic construct
  REGION 1..40: note = Description of Artificial Sequence: Synthetic polypeptide
  MOD_RES 32: note = Dbu
  MOD_RES 40: note = D-alanine
PVSTYMLTNSECLSLCNDMPITNDQKKLCSNXCQIVRQQX

SEQ ID NO: 28 - protein, 30 aa, synthetic construct
  REGION 1..30: note = Description of Artificial Sequence: Synthetic polypeptide
  MOD_RES 12: note = Nle
  MOD_RES 30: note = D-alanine
RLSECLSLCNDXPITNDQKKLCSNDCLKSX

SEQ ID NO: 29 - protein, 30 aa, synthetic construct; identical to SEQ ID NO: 25
  REGION 1..30: note = Description of Artificial Sequence: Synthetic polypeptide
  MOD_RES 12: note = Nle
  MOD_RES 25: note = Orn
  MOD_RES 30: note = D-alanine

SEQ ID NO: 30 - protein, 40 aa, synthetic construct
  REGION 1..40: note = Description of Artificial Sequence: Synthetic polypeptide
  MOD_RES 40: note = D-alanine
PVSTYMLTNSECLSLCNDMPITNDQKKLCSNDCQIVRQQX

SEQ ID NO: 31 - protein, 40 aa, synthetic construct; identical to SEQ ID NO: 27
  REGION 1..40: note = Description of Artificial Sequence: Synthetic polypeptide
  MOD_RES 32: note = Orn
  MOD_RES 40: note = D-alanine

SEQ ID NO: 32 - protein, 40 aa, synthetic construct
  REGION 1..40: note = Description of Artificial Sequence: Synthetic polypeptide
  MOD_RES 19: note = Nle
  MOD_RES 40: note = D-alanine
PVSTYMLTNSECLSLCNDXPITNDQKKLCSNNCQIVRQQX

SEQ ID NO: 33 - protein, 40 aa, synthetic construct
  REGION 1..40: note = Description of Artificial Sequence: Synthetic polypeptide
  MOD_RES 19: note = Nle
  MOD_RES 40: note = X-alanine
PVSTYMLTNSECLSLCNDXPITNDQKKLCSNKCQIVRQQX

SEQ ID NO: 34 - protein, 40 aa, synthetic construct
  REGION 1..40: note = Description of Artificial Sequence: Synthetic polypeptide
  MOD_RES 19: note = Nle
  MOD_RES 32: note = Dbu
  MOD_RES 40: note = D-alanine
PVSTYMLTNSECLSLCNDXPITNDQKKLCSNXCQIVRQQX

SEQ ID NO: 35 - protein, 40 aa, synthetic construct
  REGION 1..40: note = Description of Artificial Sequence: Synthetic polypeptide
  MOD_RES 19: note = Nle
  MOD_RES 40: note = D-alanine
PVSTYMLTNSECLSLCNDXPITNDQKKLCSNDCQIVRQQX

SEQ ID NO: 36 - protein, 40 aa, synthetic construct; identical to SEQ ID NO: 34
  REGION 1..40: note = Description of Artificial Sequence: Synthetic polypeptide
  MOD_RES 19: note = Nle
  MOD_RES 32: note = Dbu
  MOD_RES 32: note = orn
  MOD_RES 40: note = D-alanine

SEQ ID NO: 37 - protein, 30 aa, synthetic construct; identical to SEQ ID NO: 14
  REGION 1..30: note = Description of Artificial Sequence: Synthetic polypeptide
  MOD_RES 11: note = Nle
  MOD_RES 21: note = Nle
  MOD_RES 30: note = D-alanine

SEQ ID NO: 38 - protein, 30 aa, synthetic construct; identical to SEQ ID NO: 15
  REGION 1..30: note = Description of Artificial Sequence: Synthetic polypeptide
  MOD_RES 11: note = Nle
  MOD_RES 24: note = Dbu
  MOD_RES 30: note = D-alanine

SEQ ID NO: 39 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
NSECLSLCNDXPITNDQKKLCSSXCQSVRX

SEQ ID NO: 40 - protein, 30 aa, synthetic construct
  REGION 1..30: note = Description of Artificial Sequence: Synthetic polypeptide
  MOD_RES 11: note = Nle
  MOD_RES 30: note = D-alanine
NSECLSLCNDXPITNDQKKLCSSNCQSVRX

SEQ ID NO: 41 - protein, 30 aa, synthetic construct
  REGION 1..30: note = Description of Artificial Sequence: Synthetic polypeptide
  MOD_RES 11: note = Nle
  MOD_RES 30: note = D-alanine
NSECLSLCNDXPITNDQKKLCSSQCQSVRX

SEQ ID NO: 42 - protein, 30 aa, synthetic construct
  REGION 1..30: note = Description of Artificial Sequence: Synthetic polypeptide
  MOD_RES 11: note = Nle
  MOD_RES 30: note = AMIDATION
  MOD_RES 30: note = D-alanine
NSECLSLCNDXPITNDQKKLCSSSCQSVRX

SEQ ID NO: 43 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
QSECLSLCNDXPITNDQKKLCSNXCQSVRX

SEQ ID NO: 44 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
QSECLSLCNDXPITNDQKKLCSSXCQSVRX

SEQ ID NO: 45 - protein, 30 aa, synthetic construct
  REGION 1..30: note = Description of Artificial Sequence: Synthetic polypeptide
  MOD_RES 11: note = Nle
  MOD_RES 30: note = D-alanine
QSECLSLCNDXPITNDQKKLCSSNCQSVRX

SEQ ID NO: 46 - protein, 30 aa, synthetic construct
  REGION 1..30: note = Description of Artificial Sequence: Synthetic polypeptide
  MOD_RES 11: note = Nle
  MOD_RES 30: note = D-alanine
QSECLSLCNDXPITNDQKKLCSSQCQSVRX

SEQ ID NO: 47 - protein, 30 aa, synthetic construct
  REGION 1..30: note = Description of Artificial Sequence: Synthetic polypeptide
  MOD_RES 11: note = Nle
  MOD_RES 30: note = D-alanine
QSECLSLCNDXPITNDQKKLCSSSCQSVRX

SEQ ID NO: 48 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
SSECLSLCNDXPITNDQKKLCSNXCQSVRX

SEQ ID NO: 49 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
SSECLSLCNDXPITNDQKKLCSSXCQSVRX

SEQ ID NO: 50 - protein, 30 aa, synthetic construct
  REGION 1..30: note = Description of Artificial Sequence: Synthetic polypeptide
  MOD_RES 11: note = Nle
  MOD_RES 30: note = D-alanine
SSECLSLCNDXPITNDQKKLCSSNCQSVRX

SEQ ID NO: 51 - protein, 30 aa, synthetic construct
  REGION 1..30: note = Description of Artificial Sequence: Synthetic polypeptide
  MOD_RES 11: note = Nle
  MOD_RES 30: note = D-alanine
SSECLSLCNDXPITNDQKKLCSSQCQSVRX

SEQ ID NO: 52 - protein, 30 aa, synthetic construct
  REGION 1..30: note = Description of Artificial Sequence: Synthetic polypeptide
  MOD_RES 11: note = Nle
  MOD_RES 30: note = D-alanine
SSECLSLCNDXPITNDQKKLCSSSCQSVRX

SEQ ID NO: 53 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
GSECLSLCNDXPITNDQKKLCSNXCQSVRX

SEQ ID NO: 54 - protein, 30 aa, synthetic construct
  REGION 1..30: note = Description of Artificial Sequence: Synthetic polypeptide
  MOD_RES 11: note = Nle
  MOD_RES 24: note = Dbu
  MOD_RES 30: note = D-alanine
GSECLSLCNDXPITNDQKKLCSSXCQSVRX

SEQ ID NO: 55 - protein, 30 aa, synthetic construct
  REGION 1..30: note = Description of Artificial Sequence: Synthetic polypeptide
  MOD_RES 11: note = Nle
  MOD_RES 30: note = D-alanine
GSECLSLCNDXPITNDQKKLCSSNCQSVRX

SEQ ID NO: 56 - protein, 30 aa, synthetic construct
  REGION 1..30: note = Description of Artificial Sequence: Synthetic polypeptide
  MOD_RES 11: note = Nle
  MOD_RES 30: note = D-alanine
GSECLSLCNDXPITNDQKKLCSSQCQSVRX

SEQ ID NO: 57 - protein, 30 aa, synthetic construct
  REGION 1..30: note = Description of Artificial Sequence: Synthetic polypeptide
  MOD_RES 11: note = Nle
  MOD_RES 30: note = D-alanine
GSECLSLCNDXPITNDQKKLCSSSCQSVRX

SEQ ID NO: 58 - protein, 7 aa, synthetic construct
  REGION 1..7: note = Description of Artificial Sequence: Synthetic peptide
  REGION 1..7: note = repeat unit
  MOD_RES 7: note = Amino acid Xaa
IEKKIEX

SEQ ID NO: 59 - protein, 7 aa, synthetic construct
  REGION 1..7: note = Description of Artificial Sequence: Synthetic peptide
  REGION 1..7: note = repeat unit
IEKKIEG

SEQ ID NO: 60 - protein, 28 aa, synthetic construct
  REGION 1..28: note = Description of Artificial Sequence: Synthetic peptide
  REGION 1..28: note = coiled coil chain segment
IEKKIEGIEKKIEGIEKKIEGIEKKIEG

SEQ ID NO: 61 - protein, 28 aa, synthetic construct
  REGION 1..28: note = Description of Artificial Sequence: Synthetic peptide
  REGION 1..28: note = coiled coil chain segment
IEKKIEAIEKKIEAIEKKIEAIEKKIEA

SEQ ID NO: 62 - protein, 28 aa, synthetic construct
  REGION 1..28: note = Description of Artificial Sequence: Synthetic peptide
  REGION 1..28: note = coiled coil chain segment
IEKKIESIEKKIESIEKKIESIEKKIES

SEQ ID NO: 63 - protein, 14 aa, synthetic construct
  REGION 1..14: note = Description of Artificial Sequence: Synthetic peptide
  REGION 1..14: note = TT830-843
QYIKANSKFIGITE

SEQ ID NO: 64 - protein, 16 aa, synthetic construct
  REGION 1..16: note = Description of Artificial Sequence: Synthetic peptide
  REGION 1..16: note = TT1064-1079
IREDNNITLKLDRCNN

SEQ ID NO: 65 - protein, 15 aa, synthetic construct
  REGION 1..15: note = Description of Artificial Sequence: Synthetic peptide
  REGION 1..15: note = TT1084-1099
VSIDKFRIFCKANPK

SEQ ID NO: 66 - protein, 22 aa, synthetic construct
  REGION 1..22: note = Description of Artificial Sequence: Synthetic peptide
  REGION 1..22: note = TT947-968
FNNFTVSFWLRVPKVSASHLET

SEQ ID NO: 67 - protein, 16 aa, synthetic construct
  REGION 1..16: note = Description of Artificial Sequence: Synthetic peptide
  REGION 1..16: note = TT1174-1189
LKFIIKRYTPNNEIDS

SEQ ID NO: 68 - protein, 20 aa, synthetic construct
  REGION 1..20: note = Description of Artificial Sequence: Synthetic peptide
  REGION 1..20: note = DTD271-290
PVFAGANYAAWAVNVAQVID

SEQ ID NO: 69 - protein, 20 aa, synthetic construct
  REGION 1..20: note = Description of Artificial Sequence: Synthetic peptide
  REGION 1..20: note = DTD321-340
VHHNTEEIVAQSIALSSLMV

SEQ ID NO: 70 - protein, 20 aa, synthetic construct
  REGION 1..20: note = Description of Artificial Sequence: Synthetic peptide
  REGION 1..20: note = DTD331-350
QSIALSSLMVAQAIPLVGEL

SEQ ID NO: 71 - protein, 20 aa, synthetic construct
  REGION 1..20: note = Description of Artificial Sequence: Synthetic peptide
  REGION 1..20: note = DTD351-370
VDIGFAAYNFVESIINLFQV

SEQ ID NO: 72 - protein, 20 aa, synthetic construct
  REGION 1..20: note = Description of Artificial Sequence: Synthetic peptide
  REGION 1..20: note = DTD411-430
QGESGHDIKITAENTPLPIA

SEQ ID NO: 73 - protein, 20 aa, synthetic construct
  REGION 1..20: note = Description of Artificial Sequence: Synthetic peptide
  REGION 1..20: note = DTD431-450
GVLLPTIPGKLDVNKSKTHI

SEQ ID NO: 74 - protein, 20 aa, synthetic construct
  REGION 1..20: note = Description of Artificial Sequence: Synthetic peptide
  REGION 1..20: note = TT632-651
TIDKISDVSTIVPYIGPALN

SEQ ID NO: 75 - protein, 25 aa, synthetic construct
  REGION 1..25: note = Description of Artificial Sequence: Synthetic peptide
  REGION 1..25: note = CTMOMP36-60
ALNIWDRFDVFCTLGATTGYLKGNS

SEQ ID NO: 76 - protein, 14 aa, synthetic construct
  REGION 1..14: note = Description of Artificial Sequence: Synthetic peptide
  REGION 1..14: note = TraT1
GLQGKIADAVKAKG

SEQ ID NO: 77 - protein, 19 aa, synthetic construct
  REGION 1..19: note = Description of Artificial Sequence: Synthetic peptide
  REGION 1..19: note = TraT2
GLAAGLVGMAADAMVEDVN

SEQ ID NO: 78 - protein, 20 aa, synthetic construct
  REGION 1..20: note = Description of Artificial Sequence: Synthetic peptide
  REGION 1..20: note = TraT3
STETGNQHHYQTRVVSNANK

SEQ ID NO: 79 - protein, 20 aa, synthetic construct
  REGION 1..20: note = Description of Artificial Sequence: Synthetic peptide
  REGION 1..20: note = HbcAg50-69
PHHTALRQAILCWGELMTLA

SEQ ID NO: 80 - protein, 15 aa, synthetic construct
  REGION 1..15: note = Description of Artificial Sequence: Synthetic peptide
  REGION 1..15: note = HbSAg19-33
FFLLTRILTIPQSLD

SEQ ID NO: 81 - protein, 13 aa, synthetic construct
  REGION 1..13: note = Description of Artificial Sequence: Synthetic peptide
  REGION 1..13: note = HA307-319
PKYVKQNTLKLAT

SEQ ID NO: 82 - protein, 16 aa, synthetic construct
  REGION 1..16: note = Description of Artificial Sequence: Synthetic peptide
  REGION 1..16: note = MA17-31
YSGPLKAEIAQRLEDV

SEQ ID NO: 83 - protein, 20 aa, synthetic construct
  REGION 1..20: note = Description of Artificial Sequence: Synthetic peptide
  REGION 1..20: note = MVF258-277
GILESRGIKARITHVDTESY

SEQ ID NO: 84 - protein, 15 aa, synthetic construct
  REGION 1..15: note = Description of Artificial Sequence: Synthetic peptide
  REGION 1..15: note = MVF288-302
LSEIKGVIVHRLEGV

SEQ ID NO: 85 - protein, 17 aa, synthetic construct
  REGION 1..17: note = Description of Artificial Sequence: Synthetic peptide
  REGION 1..17: note = SM Th
KWFKTNAPNGVDEKIRI

SEQ ID NO: 86 - protein, 13 aa, synthetic construct
  REGION 1..13: note = Description of Artificial Sequence: Synthetic peptide
  REGION 1..13: note = PADRE1
  MOD_RES 1: note = D-alanine
  MOD_RES 13: note = D-alanine
XKFVAAWTLKAAX

SEQ ID NO: 87 - protein, 12 aa, synthetic construct
  REGION 1..12: note = Description of Artificial Sequence: Synthetic peptide
  REGION 1..12: note = PADRE2
  MOD_RES 1: note = D-alanine
  MOD_RES 2: note = cyclohexylalanine
  MOD_RES 12: note = D-alanine
XXVAAWTLKAAX

SEQ ID NO: 88 - protein, 54 aa, synthetic construct
  REGION 1..54: note = synthethic peptide moiety
GGIEKKIESIEKKIESIEKKIESIEKKIESIEKKIAKMEKASSVFNVVNSKKKC